(19) United States
(12) Patent Application Publication
(10) Pub. No.: US 2025/0129052 A1
(43) Pub. Date: Apr. 24, 2025

(54) PYRAZINE COMPOUNDS FOR THE CONTROL OF INVERTEBRATE PESTS

(71) Applicant: BASF SE, Ludwigshafen (DE)

(72) Inventors: Nikolas Huwyler, Ludwigshafen (DE); Karsten Koerber, Ludwigshafen (DE); Julia Pedroni, Ludwigshafen (DE); Erik Gilberg, Köln (DE)

(21) Appl. No.: 18/685,544

(22) PCT Filed: Aug. 16, 2022
(86) PCT No.: PCT/EP2022/072821

§ 371 Date: Feb. 22, 2024

(30) Foreign Application Priority Data
Aug. 27, 2021 (EP) 21193535.8
Aug. 27, 2021 (EP) 21193536.6
Dec. 16, 2021 (EP) 21215019.7

(51) Int. Cl. C07D 403/04; A01N 43/653; A01P 7/04; A61K 31/496; A61P 33/14; C07D 401/14

(52) U.S. Cl. CPC C07D 403/04; A01N 43/653; A01P 7/04; A61K 31/496; A61P 33/14; C07D 401/14

(57) ABSTRACT

The invention relates to compounds of formula (I) wherein the variables have the meanings as defined in the specification, to compositions comprising them, to active compound combinations comprising them, and to their use for protecting growing plants and animals from attack or infestation by invertebrate pests, furthermore, to seed comprising such compounds.

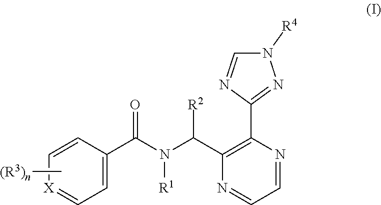

The invention relates to compounds of formula I

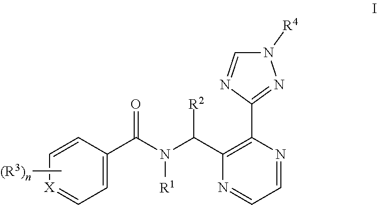

wherein
R¹ is H, OH, C₁-C₆-alkyl, C₁-C₆-haloalkyl, C₃-C₆-cycloalkyl, C₃-C₆-halocycloalkyl, C₁-C₅-alkoxy, C₂-C₄-alkenyl, C₂-C₄-alkynyl, C₁-C₄-alkyl-C₃-C₆-cycloalkyl, C₁-C₄-alkyl-C₃-C₆-halocycloalkyl, which groups are unsubstituted, or partially or fully substituted with R¹¹; or C(═N—R¹¹)R¹², C(O)R^{11a};
   R¹¹ is CN, NO₂, NR¹²R¹³, C(O)NH₂, C(S)NH₂, C(O)OH, OR¹⁴, Si(CH₃)₃; C₁-C₆-alkyl; C₁-C₆-haloalkyl; C₂-C₆-alkenyl; C₂-C₆-haloalkenyl; C₂-C₆-alkynyl; C₂-C₆-haloalkynyl; C₃-C₄-cycloalkyl-C₁-C₂-alkyl, which ring is unsubstituted or substituted with 1 or 2 halogen; 3- to 6-membered heterocyclyl, which rings are unsubstituted or substituted with R^{a}; 5- or 6-membered hetaryl, or phenyl, which rings are unsubstituted or substituted with R^{3a};
   R^{a} halogen, CN, NO₂, OH, C₁-C₄-alkyl, C₁-C₄-haloalkyl, C₁-C₄-alkoxy-C₁-C₄-alkyl, C₁-C₄-alkoxy, C₁-C₄-haloalkoxy, C₃-C₄-cycloalkyl, C₃-C₄-halocycloalkyl, S(O)ₘ-C₁-C₄-alkyl, S(O)ₘ-C₁-C₄-haloalkyl, S(O)ₘ-C₃-C₄-cycloalkyl, S(O)ₘ—C₃-C₄-halocycloalkyl, and oxo;
   R^{11a} is NR¹²R¹³, C(O)NH₂, C(S)NH₂, C(O)OH, OR¹⁴, Si(CH₃)₃; C₁-C₆-haloalkyl; C₂-C₆-alkenyl; C₂-C₆-haloalkenyl; C₂-C₆-alkynyl; C₂-C₆-haloalkynyl; C₃-C₄-cycloalkyl-C₁-C₂-alkyl, which ring is unsubstituted or substituted with 1 or 2 halogen; 3- to 6-membered heterocyclyl, which rings are unsubstituted or substituted with R^{a};
   R¹², R¹³ are independently from each other H, C₁-C₄-alkyl, C₁-C₄-alkoxy, C₁-C₄-haloalkoxy, C₁-C₄-haloalkyl, C₃-C₆-cycloalkyl, C(O)—C₁-C₄-alkyl, C(O)—C₁-C₄-haloalkyl, C(O)—C₃-C₄-cycloalkyl, C(O)—C₃-C₄-halocycloalkyl, C(O)NR¹²¹R¹³¹, S(O)ₘ—C₁-C₄-haloalkyl, S(O)ₘ—C₃-C₄-cycloalkyl, S(O)ₘ—C₃-C₄-halocycloalkyl; 3- to 6-membered heterocyclyl, which rings are unsubstituted or substituted with R^{a}; 5- or 6-membered hetaryl, or phenyl, which rings are unsubstituted or substituted with R^{3a}; or
   R¹² and R¹³, together with the nitrogen atom to which they are bound, form a 3-, 4-, 5-, 6- or 7-membered saturated, partially unsaturated or fully unsaturated heterocycle, which hetero-cycle may additionally contain 1 or 2 heteroatoms or heteroatom groups selected from N, O, and S(O)ₘ as ring members, and which heterocycle is unsubstituted or substituted with one or more substituents selected from R^{a};
      R¹²¹ and R¹³¹ are independently from each other H, C₁-C₄-alkyl, C₁-C₄-haloalkyl, C₃-C₆-cycloalkyl, C₃-C₆-halocycloalkyl, C₁-C₄-alkoxy, C₁-C₄-haloalkoxy; C₁-C₄-alkyl-phenyl, C₁-C₄-alkyl-3-6-membered hetaryl, phenyl, 3- to 6-membered heterocyclyl, which rings are unsubstituted or substituted with R^{a}; or 5- or 6-membered hetaryl which rings are unsubstituted or substituted with R^{3a}; or
      R¹²¹ and R¹³¹ together with the nitrogen atom they are bound to form a 3-6 membered saturated, partially or fully unsaturated heterocycle, which may further contain 1 or 2 heteroatoms ring members selected from N, O and S, wherein S may be oxidized, which heterocycle is unsubstituted or substituted with R^{a};
      m is 0, 1, or 2;
   R¹⁴ is H, C₁-C₄-alkyl, C₁-C₄-haloalkyl, C₃-C₆-cycloalkyl, C₃-C₆-halocycloalkyl, C₃-C₄-cycloalkyl-C₁-C₂-alkyl, C₃-C₄-halocycloalkyl-C₁-C₂-alkyl, C(O)—C₁-C₄-alkyl, C(O)—C₁-C₄-haloalkyl, C(O)—C₃-C₄-cycloalkyl, C(O)—C₃-C₄-halocycloalkyl, or phenyl which is unsubstituted or partially or fully substituted with R^{3a};
R² is H, CN, C₁-C₃-alkyl, C₁-C₃-haloalkyl, C₂-C₃-alkenyl, C₂-C₃-alkynyl;
X is CH, CR³, or N;
R³ is halogen, CN, NO₂, C₁-C₄-alkyl, C₃-C₆-cycloalkyl, C₁-C₆-haloalkyl, C₁-C₆-halocycloalkyl, OR¹⁴, S(O)ₘ—R¹⁴; which are unsubstituted or substituted with R^{3a};
   R^{3a} halogen, CN, NO₂, OH, C₁-C₄-alkyl, C₁-C₄-haloalkyl, C₁-C₄-alkoxy-C₁-C₄-alkyl, C₁-C₄-alkoxy, C₁-C₄-haloalkoxy, C₃-C₄-cycloalkyl, C₃-C₄-halocycloalkyl, S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocycloalkyl;
   n is 0, 1, 2, or 3;
R⁴ is OR¹⁴, CN, C₁-C₆-alkyl, C₃-C₆-cycloalkyl, C₁-C₆-haloalkyl, C₁-C₆-halocycloalkyl, C₂-C₄-alkenyl, C₂-C₄-halo-alkenyl, C₂-C₄-alkynyl, each unsubstituted or partially or fully substituted with R⁴¹;
   S(O)ₘ—C₁-C₄-alkyl, S(O)ₘ—C₁-C₄-haloalkyl, S(O)ₘ—C₃-C₄-cyclo¬alkyl, S(O)ₘ—C₃-C₄-halocyclo¬alkyl, NR¹²R¹³, C(O)NR¹²R¹³, C(O)OR¹⁴, 3- to 6-membered heterocyclyl, which rings are unsubstituted or substituted with R^{a}; 5- or 6-membered hetaryl, or phenyl, which rings are unsubstituted or partially or fully substituted with R³;
   R⁴¹ is H, OR¹⁵, NR¹²R¹³, C₁-C₄-alkyl, C₁-C₄-haloalkyl, C₃-C₆-cycloalkyl, C(O)—C₁-C₄-alkyl, C(O)—C₁-C₄-haloalkyl, C(O)—C₃-C₄-cycloalkyl, C(O)—C₃-C₄-halocycloalkyl, C(O)NR¹²¹R¹³¹;
      S(O)ₘ—C₁-C₄-haloalkyl, S(O)ₘ—C₃-C₄-cycloalkyl, S(O)ₘ—C₃-C₄-halocycloalkyl; 3- to 6-membered heterocyclyl, 5- or 6-membered hetaryl, or phenyl;
      which non-aromatic cyclic R⁴¹ groups are unsubstituted or partially or fully substituted with R^{a};
      which aromatic R⁴¹ groups are unsubstituted or partially or fully substituted with R^{3a};
   R¹⁵ is H, C₁-C₄-alkyl, or C₁-C₄-haloalkyl, C₃-C₆-cycloalkyl, C₁-C₆-halocycloalkyl, which carbon chains are unsubstituted or partially or fully substituted with R¹¹; or 3- to 6-membered heterocyclyl, which rings are unsubstituted or substituted with R^{a}; 5- or 6-membered hetaryl, or phenyl, which rings are unsubstituted or partially or fully substituted with R^{3a};
and the N-oxides, stereoisomers and agriculturally or veterinarily acceptable salts thereof.

The invention also provides agricultural compositions comprising at least one compound of formula I, a stereoisomer thereof and/or an agriculturally acceptable salt thereof and at least one liquid and/or solid carrier, especially at least one inert liquid and/or solid agriculturally acceptable carrier.

The invention also provides a veterinary composition comprising at least one compound of formula I, a stereoisomer thereof and/or a veterinarily acceptable salt thereof and at least one liquid and/or solid carrier, especially at least one inert veterinarily liquid and/or solid acceptable carrier.

The invention also provides a method for controlling invertebrate pests which method comprises treating the pests, their food supply, their habitat or their breeding ground or a cultivated plant, plant propagation materials (such as seed), soil, area, material or environment in which the pests are growing or may grow, or the materials, cultivated plants, plant propagation materials (such as seed), soils, surfaces or spaces to be protected from pest attack or infestation with a pesticidally effective amount of a compound of formula I or a salt thereof as defined herein.

The invention also relates to plant propagation material, in particular seed, comprising at least one compound of formula I and/or an agriculturally acceptable salt thereof.

The invention further relates to a method for treating or protecting an animal from infestation or infection by parasites which comprises bringing the animal in contact with a parasiticidally effective amount of a compound of formula I or a veterinarily acceptable salt thereof. Bringing the animal in contact with the compound I, its salt or the veterinary composition of the invention means applying or administering it to the animal.

WO 2017/192385, WO2020/070049, WO2021/037614, WO2021/122645, WO2021/068179, and WO2021/069575 describe structurally closely related active compounds. These compounds are mentioned to be useful for combating invertebrate pests.

Nevertheless, there remains a need for highly effective and versatile agents for combating invertebrate pests. It is therefore an object of the invention to provide compounds having a good pesticidal activity and showing a broad activity spectrum against a large number of different invertebrate pests, especially against difficult to control pests, such as insects.

It has been found that these objects can be achieved by compounds of formula I as depicted and defined below, and by their stereoisomers, salts, tautomers and N-oxides, in particular their agriculturally acceptable salts.

Compounds I can be obtained by alkylation of a compound II with a suitable alkylating agent Ill (e.g. alkyl halide). In formula III R¹ has the meaning as in formula I, and Y is a nucleophilic leaving group, such as a halide, preferably Br or Cl. The alkylation can be effected under standard conditions known from literature.

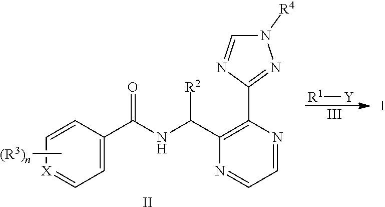

This transformation is usually carried out at temperatures of from −10° C. to +110° C., preferably from 0° C. to 25° C., in an inert solvent and in the presence of a base [cf. WO 2002100846].

The starting materials are generally reacted with one another in equimolar amounts. In terms of yield, it may be advantageous to employ an excess of Ill, based on II.

Compounds II can be obtained by reaction of an amino compound IV with a carboxylic acid V

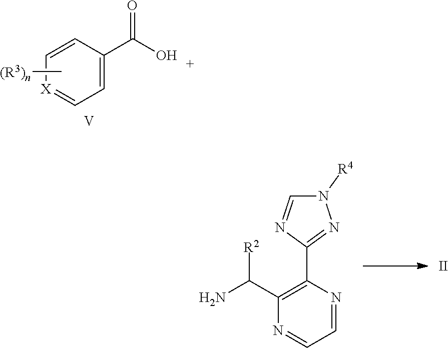

This transformation is usually carried out at temperatures of from −20° C. to 50° C., preferably from 0° C. to 25° C., in an inert solvent, in the presence of a base [cf. A. E I-Faham, Chem. Rev. 2011, 6557], or alternatively in two steps by preparation of an intermediate acyl chloride from V under conditions known from literature, e.g. by reaction with thionyl chloride or oxalyl chloride in dimethylformamide (cf. Schaefer et al, Organic Syntheses 1929, 32) followed by reaction with IV in the presence of a base, optionally under Schotten-Baumann conditions (Baumann, Chem. Ber. 1886, 3218). Suitable peptide coupling reagents are, for example, dicyclohexylcarbodiimide, diisopropylcarbodiimide, 1-ethyl-3-(3′-dimethylaminopropyl)carbodiimide hydrochloride, or chloro-N,N,N′,N′-tetramethylformamidinium hexafluorophosphate, which are commonly used together with catalytic, stoichiometric, excess amounts of additives, such as 1-hydroxybenzotriazole, 1-hydroxy-7-aza-benzotriazole, 4-(dimethylamino)pyridine, and/or 1-methylimidazole.

Suitable solvents are halogenated hydrocarbons, such as dichloromethane (DCM) or 1,2-dichloroethane, ethers, such as diethylether, tetrahydrofurane (THF) or 1,4-dioxane, or high-boiling solvents such as dimethylformamide (DMF), preferably DCM or DMF, or in aqueous media.

Suitable bases are, in general, inorganic compounds, such as alkali metal and alkaline earth metal hydroxides, such as LiOH, NaOH, KOH, or Ca(OH)₂, alkali metal and alkaline earth metal carbonates, such as Na₂CO₃, K₂CO₃, or Cs₂CO₃, alkali metal bicarbonates, such as NaHCO₃, or organic bases, e.g. tertiary amines, such as triethylamine, diisopropylethylamine, N-methylpiperidine, or basic aromatic rings, such as pyridine, 2,4,6-collidine, 2,6-lutidine, or 4-(dimethylamino)pyridine, or bicyclic amines, such as 1,8-diazabicylo[5.4.0]undec-7-ene (DBU), 1,5-diazabicyclo[4.3.0]non-5-ene (DBN), or 1,4-diazabicyclo[2.2.2]octane (DABCO).

Particular preference is given to triethylamine, diisopropylethylamine, and NaOH.

The bases are generally employed in stoichiometric or excess amounts; however, they can also be used in catalytic amounts or, if appropriate, as the solvent.

The starting materials are generally reacted with one another in equimolar amounts. In terms of yield, it may be advantageous to employ an excess of IV based on V.

Compounds IV can be obtained by reductive amination of a compound VI.

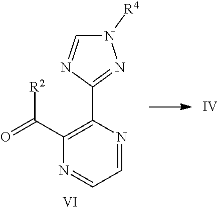

This transformation is usually carried out at temperatures of from 0° C. to 130° C., preferably from 20° C. to 70° C., generally in alcoholic and/or aqueous media and in the presence of a reagent and a reducing agent [cf. WO2021037614]. Suitable solvents are alcohols, such as methanol, ethanol, n-propanol, 2-propanol, or n-butanol, or water, preferably methanol. It is also possible to use mixtures of the aforementioned solvents. Suitable reagents are ammonium acetate (NH₄Ac), ammonium formate, NH₄OH, NH₄Cl, or ammonia. Suitable reducing agents are NaBH₃CN, sodium triacetoxyborohydride, or NaBH₄.

Preference is given to ammonium acetate and NaBH₃CN, resp.

Compounds VI are obtainable from compounds VII in a two-step sequence consisting of Stille coupling of VII with an alkoxyalkenylstannane such as VIII followed by hydrolysis of the resulting enol ether moiety to the ketone VI.

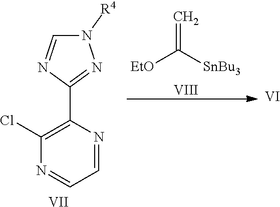

The Stille coupling reaction is usually carried out at temperatures from 50° C. to 150° C., preferably from 70° C. to 120° C., in an inert solvent in the presence of one or more catalysts and optionally in the presence of one or more additives and a base [cf. H. Lin et al., Bioorg Med Chem Lett 2010, 679]. Suitable solvents are aromatic hydrocarbons such as toluene, o-, m-, p-xylene, and mesitylene, or ethers such as THF and 1,4-dioxane, preferably toluene or 1,4-dioxane. It is also possible to use mixtures of the aforementioned solvents.

Suitable catalysts are palladium complexes, such as tetrakis(triphenylphosphine)palladium, tris(dibenzylideneacetone)dipalladium, palladium diacetate, dichloro-bis(triphenylphosphine)palladium, and [1,1′-bis(diphenylphosphino)ferrocene]dichloropalladium, preferably dichlorobis(triphenylphosphine)palladium. Further suitable optional catalysts are com-mon ligands, such as dicyclohexyl[2′,4′,6′-tris(propan-2-yl)[1,1′-biphenyl]-2-yl]phosphine or tri-phenylphosphine. Suitable additives are, in general, inorganic compounds, such as cesium fluo-ride and cuprous iodide. The starting materials are generally reacted with one another in equimolar amounts. In terms of yield, it may be advantageous to employ an excess of VIII, based on VII.

The hydrolysis is usually carried out at temperatures from −20° C. to 40° C., preferably from 0° C. to 25° C., in aqueous acidic media containing aqueous HCl at concentrations between 0.5M and 3M and optionally containing an organic solvent such as acetonitrile, acetone, THF, or methanol (cf H. Lin et al., Bioorg Med Chem Lett 2010, 679).

Compounds VII are obtainable from triazoles IX. In formula X, group Z is a leaving group, e.g. a halide, such as I, Br, and Cl, or a sulfonate, such as triflate or mesylate.

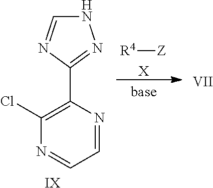

This transformation is usually carried out at temperatures from 0° C. to 100° C., preferably from 10° C. to 60° C., in an inert solvent and in the presence of a base [cf. J. Bradshaw et al., J. Heterocycl. Chem. 1986, 361]. Suitable solvents are halogenated hydrocarbons, such as DCM, 1,2-dichloroethane, or chloroform, ethers, such as diethylether, tert-butylmethylether, dioxane, or THF, nitriles, such as acetonitrile or propionitrile, alcohols, such as methanol or ethanol, and polar aprotic solvents, such as dimethyl sulfoxide (DMSO), DMF, or dimethylacetamide (DMA), preferably acetonitrile. It is also possible to use mixtures of the aforementioned solvents.

Suitable bases are, in general, inorganic compounds, such as alkali metal and alkaline earth metal hydrides, such as NaH, KH, alkali metal and alkaline earth metal carbonates, such as Na₂CO₃, K₂CO₃, or Cs₂CO₃, alkali metal bicarbonates, such as NaHCO₃, or organic bases, e.g. tertiary amines such as triethylamine or diisopropylethylamine. Preference is given to K₂CO₃.

The bases are generally employed in equimolar amounts; however, they can also be used in excess or, if appropriate, as solvent.

The starting materials are generally reacted with one another in equimolar amounts. In terms of yield, it may be advantageous to employ an excess of X, based on IX.

Triazole IX is obtainable from compound XI by reaction with 1 to 1.5 equivalents of hydrazine hydrate XII in acetic acid (AcOH) as solvent, optionally using an alcohol, such as methanol, ethanol, or 2-propanol, or an ether, such as 1,4-dioxane, as a cosolvent, at temperatures from 25° C. to 110° C., as known from literature (cf. Lin et al, J. Org. Chem. 1979, 4160; Wrobleski et al, J. Med. Chem. 2019, 8973). Alternatively, compounds VII can be directly obtained from compound XI by reaction with a substituted hydrazine of the type R⁴NH—NH₂.

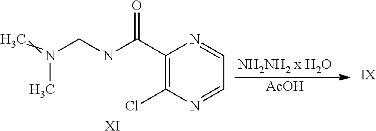

Compound XI is obtainable from commercially available 3-chloropyrazine-2-carboxamide (XII) by reaction with N,N-dimethylformamide dimethyl acetal (DMFDMA).

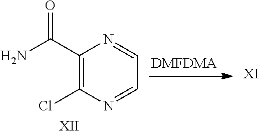

This transformation is usually carried out using 1.5 to 3 equivalents of DMFDMA at temperatures from 0° C. to 100° C., preferably from 25° C. to 90° C., in an inert solvent [cf. Lin et al, J. Org. Chem. 1979, 4160; Wrobleski et al, J. Med. Chem. 2019, 8973]. Suitable solvents are halogenated hydrocarbons, such as DCM and 1,2-dichloroethane, ethers, such as THF, aromatic solvents, such as toluene, and polar aprotic solvents, such as DMSO, preferably DCM.

The starting materials are generally reacted with one another in equimolar amounts. In terms of yield, it may be advantageous to employ an excess of DMFDMA, based on XII.

Further, compounds I can be obtained from 4-methoxybenzyl-(PMB-)substituted compounds I (R⁴=PMB), or a regioisomer of such compounds I in which the PMB group is attached to any one of the three nitrogen atoms of the triazole ring, in a two-step sequence involving removal of the PMB group by treatment with an acid such as TFA, e.g. as described in WO2011076725, followed by reaction of the intermediate (Int) using the reagents and reaction conditions as described above for the synthesis of compounds VII from compounds IX. Intermediate compounds (Int) are novel. The variables in formula (Int) are as defined for formula I.

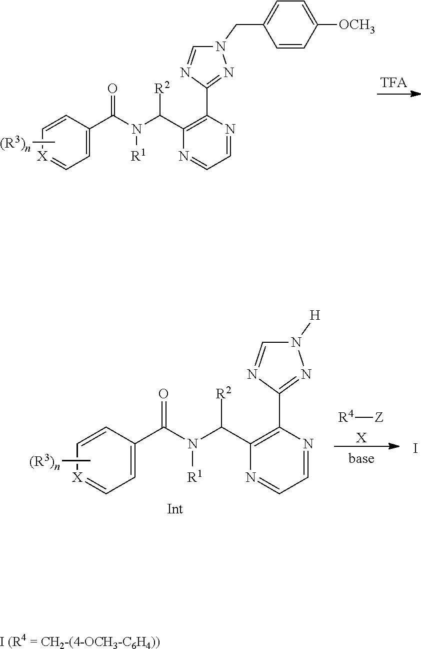

The reaction mixtures are worked up in a customary manner, e.g. by mixing with water, extracting with an appropriate organic solvent, separating the phases and, if appropriate, chromatographic purification of the crude products. Some of the intermediates and end products are obtained in the form of colourless or slightly brownish viscous oils which are purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

However, if the synthesis yields mixtures of isomers, a separation is generally not necessarily required since in some cases the individual isomers can be interconverted during work-up for use or during application (e.g. under the action of light, acids or bases). Such conversions may also take place after use, e.g. in the treatment of plants in the treated plant, or in the pest to be controlled.

The organic moieties groups mentioned in the above definitions of the variables are—like the term halogen—collective terms for individual listings of the individual group members. The prefix COₙ—Cₘ indicates in each case the possible number of carbon atoms in the group.

The term “partially or fully substituted” by a radical means that in general the group is substituted with same or different radicals.

The term “halogen” denotes in each case fluorine, bromine, chlorine, or iodine, in particular fluorine, chlorine, or bromine.

The term “alkyl” as used herein and in the alkyl moieties of alkylamino, alkylcarbonyl, alkylthio, alkylsulfinyl, alkylsulfonyl and alkoxyalkyl denotes in each case a straight-chain or branched alkyl group having usually from 1 to 10 carbon atoms, frequently from 1 to 6 carbon atoms, preferably 1 to 4 carbon atoms, more preferably from 1 to 3 carbon atoms. Examples of an alkyl group are methyl (Me), ethyl (Et), n-propyl (n-Pr), iso-propyl, n-butyl, 2-butyl, iso-butyl, tert-butyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, and 1-ethyl-2-methylpropyl.

The term “haloalkyl” as used herein and in the haloalkyl moieties of haloalkylcarbonyl, haloalkoxycarbonyl, haloalkylthio, haloalkylsulfonyl, haloalkylsulfinyl, haloalkoxy and haloalkoxyalkyl, denotes in each case a straight-chain or branched alkyl group having usually from 1 to 10 carbon atoms, frequently from 1 to 6 carbon atoms, preferably from 1 to 4 carbon atoms, wherein the hydrogen atoms of this group are partially or totally replaced with halogen atoms. Preferred haloalkyl moieties are selected from C₁-C₄-haloalkyl, more preferably from C₁-C₃-haloalkyl or C₁-C₂-haloalkyl, in particular from C₁-C₂-fluoroalkyl such as fluoromethyl, difluoromethyl, trifluoromethyl, 1-fluoroethyl, 2-fluoroethyl, 2,2-difluoroethyl, 2,2,2-trifluoroethyl, pentafluoroethyl, and the like.

The term “alkoxy” as used herein denotes in each case a straight-chain or branched alkyl group which is bonded via an oxygen atom and has usually from 1 to 10 carbon atoms, frequently from 1 to 6 carbon atoms, preferably 1 to 4 carbon atoms. Examples of an alkoxy group are methoxy, ethoxy, n-propoxy, iso-propoxy, n-butyloxy, 2-butyloxy, iso-butyloxy, tert.-butyloxy, and the like.

The term “alkoxyalkyl” as used herein refers to alkyl usually comprising 1 to 10, frequently 1 to 4, preferably 1 to 2 carbon atoms, wherein 1 carbon atom carries an alkoxy radical usually comprising 1 to 4, preferably 1 or 2 carbon atoms as defined above. Examples are CH₂OCH₃, CH₂—OC₂H₅, 2-(methoxy)ethyl, and 2-(ethoxy)ethyl.

The term “haloalkoxy” as used herein denotes in each case a straight-chain or branched alkoxy group having from 1 to 10 carbon atoms, frequently from 1 to 6 carbon atoms, preferably 1 to 4 carbon atoms, wherein the hydrogen atoms of this group are partially or totally replaced with halogen atoms, in particular fluorine atoms. Preferred haloalkoxy moieties include C₁-C₄-haloalkoxy, in particular C₁-C₂-fluoroalkoxy, such as fluoromethoxy, difluoromethoxy, trifluoromethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoro-ethoxy, 2,2dichloro-2-fluorethoxy, 2,2,2-trichloroethoxy, pentafluoroethoxy and the like.

The term “alkylthio “(alkylsulfanyl: S-alkyl)” as used herein refers to a straight-chain or branched saturated alkyl group having 1 to 10 carbon atoms, preferably 1 to 4 carbon atoms (═C₁-C₄-alkylthio), more preferably 1 to 3 carbon atoms, which is attached via a sulfur atom.

The term “haloalkylthio” as used herein refers to an alkylthio group as mentioned above wherein the hydrogen atoms are partially or fully substituted by fluorine, chlorine, bromine and/or iodine.

The term “alkylsulfinyl” (alkylsulfoxyl: S(═O)-alkyl), as used herein refers to a straight-chain or branched saturated alkyl group (as mentioned above) having 1 to 10 carbon atoms, preferably 1 to 4 carbon atoms (═C₁-C₄-alkylsulfinyl), more preferably 1 to 3 carbon atoms bonded through the sulfur atom of the sulfinyl group at any position in the alkyl group.

The term “haloalkylsulfinyl” as used herein refers to an alkylsulfinyl group as mentioned above wherein the hydrogen atoms are partially or fully substituted by fluorine, chlorine, bromine and/or iodine.

The term “alkylsulfonyl” (S(═O)₂-alkyl) as used herein refers to a straight-chain or branched saturated alkyl group having 1 to 10 carbon atoms, preferably 1 to 4 carbon atoms (═C₁-C₄-alkylsulfonyl), preferably 1 to 3 carbon atoms, which is bonded via the sulfur atom of the sulfonyl group at any position in the alkyl group.

The term “haloalkylsulfonyl” as used herein refers to an alkylsulfonyl group as mentioned above wherein the hydrogen atoms are partially or fully substituted by fluorine, chlorine, bromine and/or iodine.

The term “alkylcarbonyl” refers to an alkyl group as defined above, which is bonded via the carbon atom of a carbonyl group (C═O) to the remainder of the molecule.

The term “haloalkylcarbonyl” refers to an alkylcarbonyl group as mentioned above, wherein the hydrogen atoms are partially or fully substituted by fluorine, chlorine, bromine and/or iodine.

The term “alkoxycarbonyl” refers to an alkylcarbonyl group as defined above, which is bonded via an oxygen atom to the remainder of the molecule.

The term “haloalkoxycarbonyl” refers to an alkoxycarbonyl group as mentioned above, wherein the hydrogen atoms are partially or fully substituted by fluorine, chlorine, bromine and/or iodine.

The term “alkenyl” as used herein denotes in each case a singly unsaturated hydrocarbon radical having usually 2 to 10, frequently 2 to 6, preferably 2 to 4 carbon atoms, e.g. vinyl, allyl (2-propen-1-yl), 1-propen-1-yl, 2-propen-2-yl, methallyl (2-methylprop-2-en-1-yl), 2-buten-1-yl, 3-buten-1-yl, 2-penten-1-yl, 3-penten-1-yl, 4-penten-1-yl, 1-methylbut-2-en-1-yl, 2-ethylprop-2-en-1-yl and the like.

The term “haloalkenyl” as used herein refers to an alkenyl group as defined above, wherein the hydrogen atoms are partially or totally replaced with halogen atoms.

The term “alkynyl” as used herein denotes in each case a singly unsaturated hydrocarbon radical having usually 2 to 10, frequently 2 to 6, preferably 2 to 4 carbon atoms, e.g. ethynyl, propargyl (2-propyn-1-yl), 1-propyn-1-yl, 1-methylprop-2-yn-1-yl), 2-butyn-1-yl, 3-butyn-1-yl, 1-pentyn-1-yl, 3-pentyn-1-yl, 4-pentyn-1-yl, 1-methylbut-2-yn-1-yl, 1-ethylprop-2-yn-1-yl and the like.

The term “haloalkynyl” as used herein refers to an alkynyl group as defined above, wherein the hydrogen atoms are partially or totally replaced with halogen atoms.

The term “cycloalkyl” as used herein and in the cycloalkyl moieties of cycloalkoxy and cycloalkylthio denotes in each case a monocyclic cycloaliphatic radical having usually from 3 to 10 or from 3 to 6 carbon atoms, such as cyclopropyl (cC₃H₅), cyclobutyl (cC₄H₇), cyclopentyl (cC₅H₉), cyclohexyl (cC₆H₁₁), cycloheptyl, cyclooctyl, cyclononyl and cyclodecyl or cyclopropyl, cyclobutyl, cyclopentyl and cyclohexyl.

The term “halocycloalkyl” as used herein and in the halocycloalkyl moieties of halocycloalkoxy and halocycloalkylthio denotes in each case a monocyclic cycloaliphatic radical having usually from 3 to 10 C atoms or 3 to 6 C atoms, wherein at least one, e.g. 1, 2, 3, 4 or 5 of the hydrogen atoms, are replaced by halogen, in particular by fluorine or chlorine. Examples are 1- and 2-fluorocyclopropyl, 1,2-, 2,2- and 2,3-difluorocyclopropyl, 1,2,2-trifluorocyclopropyl, 2,2,3,3-tetrafluorocyclpropyl, 1- and 2-chlorocyclopropyl, 1,2-, 2,2- and 2,3-dichlorocyclopropyl, 1,2,2-trichlorocyclopropyl, 2,2,3,3-tetrachlorocyclpropyl, 1-,2- and 3-fluorocyclopentyl, 1,2-, 2,2-, 2,3-, 3,3-, 3,4-, 2,5-difluorocyclopentyl, 1-,2- and 3-chlorocyclopentyl, 1,2-, 2,2-, 2,3-, 3,3-, 3,4-, 2,5-dichlorocyclopentyl and the like.

The term “cycloalkenyl” as used herein and in the cycloalkenyl moieties of cycloalkenyloxy and cycloalkenylthio denotes in each case a mono- or bicyclic, preferably monocyclic, singly unsaturated non-aromatic radical having usually from 3 to 10, e.g. 3 or 4 or from 5 to 10 carbon atoms, preferably from 3- to 8 carbon atoms. Examples are cyclopenten-1-yl, and cyclohexen-1-yl.

The term “halocycloalkenyl” as used herein and in the halocycloalkenyl moieties of halocycloalkenyloxy and halocycloalkenylthio denotes in each case a monocyclic singly unsaturated nonaromatic radical having usually from 3 to 10, e.g. 3 or 4 or from 5 to 10 carbon atoms, preferably from 3- to 8 carbon atoms, wherein at least one, e.g. 1, 2, 3, 4 or 5 of the hydrogen atoms, are replaced by halogen, in particular by fluorine or chlorine. Examples are 3,3-difluorocyclopropen-1-yl and 3,3-dichlorocyclopropen-1-yl.

The term “cycloalkenylalkyl” refers to a cycloalkenyl group as defined above which is bonded via an alkyl group, such as a C₁-C₅-alkyl group or a C₁-C₄-alkyl group, in particular a methyl group (=cycloalkenylmethyl), to the remainder of the molecule.

The term “carbocycle” or “carbocyclyl” includes in general a 3- to 12-membered, preferably a 3- to 8-membered or a 5- to 8-membered, more preferably a 5- or 6-membered mono-cyclic, non-aromatic ring comprising 3 to 12, preferably 3 to 8 or 5 to 8, more preferably 5 or 6 carbon atoms. Preferably, the term “carbocycle” covers cycloalkyl and cycloalkenyl groups as defined above.

The term “heterocycle” or “heterocyclyl” includes in general 3- to 12-membered, preferably 3- to 6-membered, in particular 6-membered monocyclic heterocyclic non-aromatic radicals. The heterocyclic non-aromatic radicals usually comprise 1, 2, 3, 4 or 5, preferably 1, 2 or 3 heteroatoms selected from N, O, and S as ring members, wherein S-atoms as ring members may be present as S, SO, or SO₂. Examples of 5- or 6-membered heterocyclic radicals comprise saturated or unsaturated, non-aromatic heterocyclic rings, such as oxiranyl, oxetanyl, thietanyl, thietanyl-S-oxid (S-oxothietanyl), thietanyl-S-dioxid (S-dioxothiethanyl), pyrrolidinyl, pyrrolinyl, pyrazolinyl, tetrahydrofuranyl, dihydrofuranyl, 1,3-dioxolanyl, thiolanyl, S-oxothiolanyl, S-dioxothiolanyl, dihydrothienyl, S-oxodihydrothienyl, S-dioxodihydrothienyl, oxazolidinyl, oxazolinyl, thiazolinyl, oxathiolanyl, piperidinyl, piperazinyl, pyranyl, dihydropyranyl, tetrahydropyranyl, 1,3- and 1,4-dioxanyl, thiopyranyl, S. oxothiopyranyl, S-dioxothiopyranyl, dihydrothiopyranyl, S-oxodihydrothiopyranyl, S-dioxodihydrothiopyranyl, tetrahydrothiopyranyl, S-oxotetrahydrothiopyranyl, S-dioxotetrahydrothiopyranyl, morpholinyl, thiomorpholinyl, S-oxothiomorpholinyl, S-dioxothiomorpholinyl, thiazinyl and the like. Examples for heterocyclic ring also comprising 1 or 2 carbonyl groups as ring members comprise pyrrolidin-2-onyl, pyrrolidin-2,5-dionyl, imidazolidin-2-onyl, oxazolidin-2-onyl, thiazolidin-2-only, and the like.

The term “hetaryl” includes monocyclic 5- or 6-membered heteroaromatic radicals comprising as ring members 1, 2, 3 or 4 heteroatoms selected from N, O, and S. Examples of 5- or 6-membered heteroaromatic radicals include pyridyl, i.e. 2-, 3-, or 4-pyridyl, pyrimidinyl, i.e. 2-, 4- or 5-pyrimidinyl, pyrazinyl, pyridazinyl, i.e. 3- or 4-pyridazinyl, thienyl, i.e. 2- or 3-thienyl, furyl, i.e. 2- or 3-furyl, pyrrolyl, i.e. 2- or 3-pyrrolyl, oxazolyl, i.e. 2-, 3- or 5-oxazolyl, isoxazolyl, i.e. 3-, 4- or 5-isoxazolyl, thiazolyl, i.e. 2-, 3- or 5-thiazolyl, isothiazolyl, i.e. 3-, 4- or 5-isothiazolyl, pyrazolyl, i.e. 1-, 3-, 4- or 5-pyrazolyl, i.e. 1-, 2-, 4- or 5-imidazolyl, oxadiazolyl, e.g. 2- or 5-[1,3,4]oxadiazolyl, 4- or 5-(1,2,3-oxadiazol)yl, 3- or 5-(1,2,4-oxadiazol)yl, 2- or 5-(1,3,4-thiadiazol)yl, thiadiazolyl, e.g. 2- or 5-(1,3,4-thiadiazol)yl, 4- or 5-(1,2,3-thiadiazol)yl, 3- or 5-(1,2,4-thiadiazol)yl, triazolyl, e.g. 1H-, 2H- or 3H-1,2,3-triazol-4-yl, 2H-triazol-3-yl, 1H-, 2H-, or 4H-1,2,4-triazolyl and tetrazolyl, i.e. 1H- or 2H-tetrazolyl. The term “hetaryl” also includes bicyclic 8 to 10-membered heteroaromatic radicals comprising as ring members 1, 2 or 3 heteroatoms selected from N, O, and S, wherein a 5- or 6-membered heteroaromatic ring is fused to a phenyl ring or to a 5- or 6-membered heteroaromatic radical. Examples of a 5- or 6-membered heteroaromatic ring fused to a phenyl ring or to a 5- or 6-membered heteroaromatic radical include benzofuranyl, benzothienyl, indolyl, indazolyl, benzimidazolyl, benzoxathiazolyl, benzoxadiazolyl, benzothiadiazolyl, benzoxazinyl, chinolinyl, isochinolinyl, purinyl, 1,8-naphthyridyl, pteridyl, pyrido[3,2-d]pyrimidyl or pyridoimidazolyl and the like. These fused hetaryl radicals may be bonded to the remainder of the molecule via any ring atom of 5- or 6-membered heteroaromatic ring or via a carbon atom of the fused phenyl moiety.

The terms “heterocyclylalkyl” and “hetarylalkyl” refer to heterocyclyl or hetaryl, respectively, as defined above which are bonded via a C₁-C₅-alkyl group or a C₁-C₄-alkyl group, in particular a methyl group (=heterocyclylmethyl or hetarylmethyl, respectively), to the remainder of the molecule.

The term “arylalkyl” and “phenylalkyl” refer to aryl as defined above and phenyl, respectively, which are bonded via C₁-C₅-alkyl group or a C₁-C₄-alkyl group, in particular a methyl group (=arylmethyl or phenylmethyl), to the remainder of the molecule, examples including benzyl, 1-phenylethyl, 2-phenylethyl, 2-phenoxyethyl etc.

The terms “alkylene”, “cycloalkylene”, “heterocycloalkylene”, “alkenylene”, “cycloalkenylene”, “heterocycloalkenylene” and “alkynylene” refer to alkyl, cycloalkyl, heterocycloalkyl, alkenyl, cycloalkenyl, heterocycloalkenyl and alkynyl as defined above, respectively, which are bonded to the remainder of the molecule, via two atoms, preferably via two carbon atoms, of the respective group, so that they represent a linker between two moieties of the molecule.

In a particular embodiment, the variables of the compounds of the formula I have the following meanings, these meanings, both on their own and in combination with one another, being particular embodiments of the compounds of the formula I.

Embodiments and preferred compounds of the invention for use in pesticidal methods and for insecticidal application purposes are outlined in the following paragraphs.

With respect to the variables, the particularly preferred embodiments of the intermediates correspond to those of the compounds of the formula I.

In a preferred embodiment, the compounds I are present in form of a mixture of compounds I.A and I.B, wherein compound I.A with S-configuration of the carbon atom neighboring the nitrogen is present in an amount of more than 50% by weight, in particular of at least 70% by weight, more particularly of at least 85% by weight, specifically of at least 90% by weight, based on the total weight of compounds I.A and I.B.

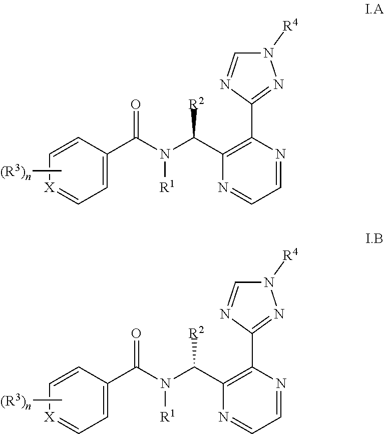

In one particularly preferred embodiment of the invention, the method comprises the step of contacting the plant, parts of it, its propagation material, the pests, their food supply, habitat or breeding grounds with a pesticidally effective amount of a compound of formula I.A.

Preferably R¹ is H, C₁-C₆-alkyl, C₃-C₆-alkynyl, C₃-C₆-cycloalkyl, or C₁-C₄-alkyl-C₃-C₆-cycloalkyl.

Preferably R² is CH₃.

X is preferably CH or CR³, particularly CH. Such compounds correspond to Formula I.1

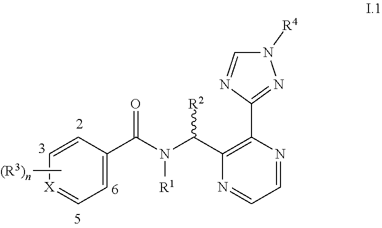

In another embodiment X is N. Such compounds correspond to formula I.2.

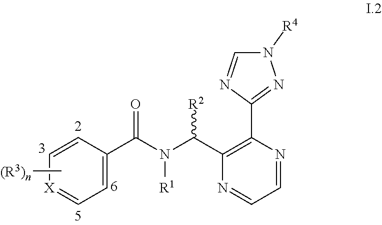

R³ is preferably halogen, CN, C₁-C₄-haloalkyl, C₁-C₄-haloalkoxy, C₃-C₄-cycloalkyl unsubstituted or substituted with one or more CN, C₃-C₄-halocycloalkyl, S(O)ₘ—C₁-C₄-alkyl, S(O)ₘ—C₁-C₄-haloalkyl, S(O)ₘ—C₃-C₄-cyclo¬alkyl, S(O)ₘ—C₃-C₄-halocyclo¬alkyl. Index m in R³ is preferably 2.

Index n is preferably 2.

In another embodiment R³ is preferably halogen, CN, C₁-C₄-haloalkyl, C₁-C₄-haloalkoxy, C₃-C₄-cycloalkyl unsubstituted or substituted with one or more R^{3a}, wherein R^{3a} is preferably CN, OH, C₁-C₄-alkoxy. Index m in R³ is preferably 2. Index n is preferably 2.

R³ groups stand preferably in positions 3 and 5.

In another embodiment R³ is preferably halogen, CN, C₁-C₄-haloalkyl, C₁-C₄-haloalkoxy, C₃-C₄-cycloalkyl, C₃-C₄-halocycloalkyl, S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocycloalkyl, or
S(O)ₘ—R¹⁴, wherein R¹⁴ is phenyl, which is partially substituted with R^{3a}.

In another embodiment of formula I compounds R³ is halogen, CN, NO₂, C₁-C₄-alkyl, C₃-C₆-cycloalkyl, C₁-C₆-haloalkyl, C₁-C₆-halocycloalkyl, OR¹⁴, S(O)ₘ—R¹⁴; wherein rings are unsubstituted or substituted with R¹¹.

R⁴ is preferably C₁-C₄-alkyl, C₁-C₄-haloalkyl, C₁-C₄-alkoxy-C₁-C₄-alkyl, CH₂C(O)NH—C₁-C₆-alkyl, S(O)ₘ—C₁-C₄-alkyl, or phenyl unsubstituted or substituted with one or more groups R³.

In particular with a view to their use, preference is given to the compounds of formula I compiled in the tables below, which compounds correspond to formula I.1*, and I.2*, respEach of the groups mentioned for a substituent in the tables is furthermore per se, independently of the combination in which it is mentioned, a particularly preferred aspect of the substituent in question.

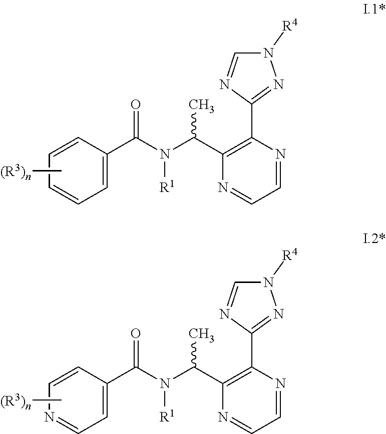

### Table 1

Compounds of formula I.1* in which R⁴ is CN, and the combination of R¹ and (R³)ₙ for a compound corresponds in each case to one row of Table A

### Table 2

Compounds of formula I.1* in which R⁴ is CH₃, and the combination of R¹ and (R³)ₙ for a compound corresponds in each case to one row of Table A

### Table 3

Compounds of formula I.1* in which R⁴ is C₂H₅, and the combination of R¹ and (R³)ₙ for a compound corresponds in each case to one row of Table A

### Table 4

Compounds of formula I.1* in which R⁴ is CH₂CH₂CH₃, and the combination of R¹ and (R³)ₙ for a compound corresponds in each case to one row of Table A

### Table 5

Compounds of formula I.1* in which R⁴ is CH(CH₃)₂, and the combination of R¹ and (R³)ₙ for a compound corresponds in each case to one row of Table A

### Table 6

Compounds of formula I.1* in which R⁴ is cC₃H₅, and the combination of R¹ and (R³)ₙ for a compound corresponds in each case to one row of Table A

### Table 7

Compounds of formula I.1* in which R⁴ is CH₂CF₃, and the combination of R¹ and (R³)ₙ for a compound corresponds in each case to one row of Table A

### Table 8

Compounds of formula I.1* in which R⁴ is CH₂OCH₃, and the combination of R¹ and (R³)ₙ for a compound corresponds in each case to one row of Table A

### Table 9

Compounds of formula I.1* in which R⁴ is C(═O)NH₂, and the combination of R¹ and (R³)ₙ for a compound corresponds in each case to one row of Table A

### Table 10

Compounds of formula I.1* in which R⁴ is C(═O)NHCH₃, and the combination of R¹ and (R³)ₙ for a compound corresponds in each case to one row of Table A

### Table 11

Compounds of formula I.1* in which R⁴ is C(═O)NHC₂H₅, and the combination of R¹ and (R³)ₙ for a compound corresponds in each case to one row of Table A

### Table 12

Compounds of formula I.1* in which R⁴ is C(═O)N(CH₃)₂, and the combination of R¹ and (R³)ₙ for a compound corresponds in each case to one row of Table A

### Table 13

Compounds of formula I.1* in which R⁴ is C(═O)N(CH₃)C₂H₅, and the combination of R¹ and (R³)ₙ for a compound corresponds in each case to one row of Table A

### Table 14

Compounds of formula I.1* in which R⁴ is C(═O)OCH₃, and the combination of R¹ and (R³)ₙ for a compound corresponds in each case to one row of Table A

### Table 15

Compounds of formula I.1* in which R⁴ is C(═O)OC₂H₅, and the combination of R¹ and (R³)ₙ for a compound corresponds in each case to one row of Table A

### Table 16

Compounds of formula I.1* in which R⁴ is CH₂C(═O)NHCH₃, and the combination of R¹ and (R³)ₙ for a compound corresponds in each case to one row of Table A

### Table 17

Compounds of formula I.1* in which R⁴ is 4,5-dihydrooxazol-2-yl, and the combination of R¹ and (R³)ₙ for a compound corresponds in each case to one row of Table A

### Table 18

Compounds of formula I.1* in which R⁴ is SO₂CH₃, and the combination of R¹ and (R³)ₙ for a compound corresponds in each case to one row of Table A

### Table 19

Compounds of formula I.1* in which R⁴ is SO₂C₂H₅, and the combination of R¹ and (R³)ₙ for a compound corresponds in each case to one row of Table A

### Table 20

Compounds of formula I.2* in which R⁴ is CN, and the combination of R¹ and (R³)ₙ for a compound corresponds in each case to one row of Table A

### Table 21

Compounds of formula I.2* in which R⁴ is CH₃, and the combination of R¹ and (R³)ₙ for a compound corresponds in each case to one row of Table A

### Table 22

Compounds of formula I.2* in which R⁴ is C₂H₅, and the combination of R¹ and (R³)ₙ for a compound corresponds in each case to one row of Table A

### Table 23

Compounds of formula I.2* in which R⁴ is CH₂CH₂CH₃, and the combination of R¹ and (R³)ₙ for a compound corresponds in each case to one row of Table A

### Table 24

Compounds of formula I.2* in which R⁴ is CH(CH₃)₂, and the combination of R¹ and (R³)ₙ for a compound corresponds in each case to one row of Table A

### Table 25

Compounds of formula I.2* in which R⁴ is cC₃H₅, and the combination of R¹ and (R³)ₙ for a compound corresponds in each case to one row of Table A

### Table 26

Compounds of formula I.2* in which R⁴ is CH₂CF₃, and the combination of R¹ and (R³)ₙ for a compound corresponds in each case to one row of Table A

### Table 27

Compounds of formula I.2* in which R⁴ is CH₂OCH₃, and the combination of R¹ and (R³)ₙ for a compound corresponds in each case to one row of Table A

### Table 28

Compounds of formula I.2* in which R⁴ is C(═O)NH₂, and the combination of R¹ and (R³)ₙ for a compound corresponds in each case to one row of Table A

### Table 29

Compounds of formula I.2* in which R⁴ is C(═O)NHCH₃, and the combination of R¹ and (R³)ₙ for a compound corresponds in each case to one row of Table A

### Table 30

Compounds of formula I.2* in which R⁴ is C(═O)NHC₂H₅, and the combination of R¹ and (R³)ₙ for a compound corresponds in each case to one row of Table A

### Table 31

Compounds of formula I.2* in which R⁴ is C(═O)N(CH₃)₂, and the combination of R¹ and (R³)ₙ for a compound corresponds in each case to one row of Table A

### Table 32

Compounds of formula I.2* in which R⁴ is C(═O)N(CH₃)C₂H₅, and the combination of R¹ and (R³)ₙ for a compound corresponds in each case to one row of Table A

### Table 33

Compounds of formula I.2* in which R⁴ is C(═O)OCH₃, and the combination of R¹ and (R³)ₙ for a compound corresponds in each case to one row of Table A

### Table 34

Compounds of formula I.2* in which R⁴ is C(═O)OC₂H₅, and the combination of R¹ and (R³)ₙ for a compound corresponds in each case to one row of Table A

### Table 35

Compounds of formula I.2* in which R⁴ is CH₂C(═O)NHCH₃, and the combination of R¹ and (R³)ₙ for a compound corresponds in each case to one row of Table A

### Table 36

Compounds of formula I.2* in which R⁴ is 4,5-dihydrooxazol-2-yl, and the combination of R¹ and (R³)ₙ for a compound corresponds in each case to one row of Table A

### Table 37

Compounds of formula I.2* in which R⁴ is SO₂CH₃, and the combination of R¹ and (R³)ₙ for a compound corresponds in each case to one row of Table A

### Table 38

Compounds of formula I.2* in which R⁴ is SO₂C₂H₅, and the combination of R¹ and (R³)ₙ for a compound corresponds in each case to one row of Table A

TABLE A

| No. | R¹ | (R³)ₙ |
|---|---|---|
| A-1 | H | 3,5-F₂ |
| A-2 | H | 3,5-Cl₂ |
| A-3 | H | 3,5-Br₂ |
| A-4 | H | 3,5-I₂ |
| A-5 | H | 3,5-(CF₃)₂ |
| A-6 | H | 3-Cl,5-F |
| A-7 | H | 3-Cl,5-Br |
| A-8 | H | 3-Cl,5-I |
| A-9 | H | 3-F,5-CN |
| A-10 | H | 3-Cl,5-CN |
| A-11 | H | 3-CF₃,5-CN |
| A-12 | H | 3-F,5-CF₃ |
| A-13 | H | 3-Cl,5-CF₃ |
| A-14 | H | 3-Br,5-CF₃ |
| A-15 | H | 3-I,5-CF₃ |
| A-16 | H | 3-Cl,5-SO₂CH₃ |
| A-17 | H | 3-Cl,5-SO₂CF₃ |
| A-18 | H | 3-Cl,5-cC₃H₅ |
| A-19 | H | 3-Cl,5-[2,2-Cl₂- |
| | | cC₃H₃] |
| A-20 | H | 3-Cl,5-OCF₃ |
| A-21 | H | 3-Br,5-OCF₃ |
| A-22 | H | 3-F,5-cC₃H₅ |
| A-23 | H | 3-Cl,5-CH₂CN |
| A-24 | H | 3-Cl,5- |
| | | C(CH₃)₂CN |
| A-25 | H | 3-CF₃,5-CH₂CN |
| A-26 | H | 3-CF₃,5- |
| | | C(CH₃)₂CN |
| A-27 | H | 3-CF₃,5-SO₂CH₃ |
| A-28 | H | 3-CF₃,5-SO₂CF₃ |
| A-29 | H | 3-CF₃,5-OCF₃ |
| A-30 | H | 3-CF₃,5-cC₃H₅ |
| A-31 | H | 3-Cl,5-[(1- |
| | | CN)cC₃H₄] |
| A-32 | H | 3-CF₃,5-[(1- |
| | | CN)cC₃H₄] |
| A-33 | H | 3-CF₃,5-[(2,2- |
| | | Cl₂)-cC₃H₃] |
| A-34 | H | 3-OCF₃,5-cC₃H₅ |
| A-35 | H | 3,5-SO₂CH₃ |
| A-36 | H | 3,5-SO₂CF₃ |
| A-37 | H | 3-Cl,5-OC₆H₅ |
| A-38 | H | 3-CF₃,5-OC₆H₅ |
| A-39 | H | 3-CF₃,5-[O-(4- |
| | | Cl-C₆H₄)] |
| A-40 | CH₃ | 3,5-F₂ |
| A-41 | CH₃ | 3,5-Cl₂ |
| A-42 | CH₃ | 3,5-Br₂ |
| A-43 | CH₃ | 3,5-I₂ |
| A-44 | CH₃ | 3,5-(CF₃)₂ |
| A-45 | CH₃ | 3-Cl,5-F |
| A-46 | CH₃ | 3-Cl,5-Br |
| A-47 | CH₃ | 3-Cl,5-I |
| A-48 | CH₃ | 3-F,5-CN |
| A-49 | CH₃ | 3-Cl,5-CN |
| A-50 | CH₃ | 3-CF₃,5-CN |
| A-51 | CH₃ | 3-F,5-CF₃ |
| A-52 | CH₃ | 3-Cl,5-CF₃ |
| A-53 | CH₃ | 3-Br,5-CF₃ |
| A-54 | CH₃ | 3-I,5-CF₃ |
| A-55 | CH₃ | 3-Cl,5-SO₂CH₃ |
| A-56 | CH₃ | 3-Cl,5-SO₂CF₃ |
| A-57 | CH₃ | 3-Cl,5-cC₃H₅ |
| A-58 | CH₃ | 3-Cl,5-[2,2-Cl₂- |
| | | cC₃H₃] |
| A-59 | CH₃ | 3-Cl,5-OCF₃ |
| A-60 | CH₃ | 3-Br,5-OCF₃ |
| A-61 | CH₃ | 3-F,5-cC₃H₅ |
| A-62 | CH₃ | 3-Cl,5-CH₂CN |
| A-63 | CH₃ | 3-Cl,5- |
| | | C(CH₃)₂CN |
| A-64 | CH₃ | 3-CF₃,5-CH₂CN |
| A-65 | CH₃ | 3-CF₃,5- |
| | | C(CH₃)₂CN |
| A-66 | CH₃ | 3-CF₃,5-SO₂CH₃ |
| A-67 | CH₃ | 3-CF₃,5-SO₂CF₃ |
| A-68 | CH₃ | 3-CF₃,5-OCF₃ |
| A-69 | CH₃ | 3-CF₃,5-cC₃H₅ |
| A-70 | CH₃ | 3-Cl,5-[(1- |
| | | CN)cC₃H₄] |
| A-71 | CH₃ | 3-CF₃,5-[(1- |
| | | CN)cC₃H₄] |
| A-72 | CH₃ | 3-CF₃,5-[(2,2- |
| | | Cl₂)-cC₃H₃] |
| A-73 | CH₃ | 3-OCF₃,5-cC₃H₅ |
| A-74 | CH₃ | 3,5-SO₂CH₃ |
| A-75 | CH₃ | 3,5-SO₂CF₃ |
| A-76 | CH₃ | 3-Cl,5-OC₆H₅ |
| A-77 | CH₃ | 3-CF₃,5-OC₆H₅ |
| A-78 | CH₃ | 3-CF₃,5-[O-(4- |
| | | Cl-C₆H₄)] |
| A-79 | C₂H₅ | 3,5-F₂ |
| A-80 | C₂H₅ | 3,5-Cl₂ |
| A-81 | C₂H₅ | 3,5-Br₂ |
| A-82 | C₂H₅ | 3,5-I₂ |
| A-83 | C₂H₅ | 3,5-(CF₃)₂ |
| A-84 | C₂H₅ | 3-Cl,5-F |
| A-85 | C₂H₅ | 3-Cl,5-Br |
| A-86 | C₂H₅ | 3-Cl,5-I |
| A-87 | C₂H₅ | 3-F,5-CN |
| A-88 | C₂H₅ | 3-Cl,5-CN |
| A-89 | C₂H₅ | 3-CF₃,5-CN |
| A-90 | C₂H₅ | 3-F,5-CF₃ |
| A-91 | C₂H₅ | 3-Cl,5-CF₃ |
| A-92 | C₂H₅ | 3-Br,5-CF₃ |
| A-93 | C₂H₅ | 3-I,5-CF₃ |
| A-94 | C₂H₅ | 3-Cl,5-SO₂CH₃ |
| A-95 | C₂H₅ | 3-Cl,5-SO₂CF₃ |
| A-96 | C₂H₅ | 3-Cl,5-cC₃H₅ |
| A-97 | C₂H₅ | 3-Cl,5-[2,2-Cl₂- |
| | | cC₃H₃] |
| A-98 | C₂H₅ | 3-Cl,5-OCF₃ |
| A-99 | C₂H₅ | 3-Br,5-OCF₃ |
| A-100 | C₂H₅ | 3-F,5-cC₃H₅ |
| A-101 | C₂H₅ | 3-Cl,5-CH₂CN |
| A-102 | C₂H₅ | 3-Cl,5- |
| | | C(CH₃)₂CN |
| A-103 | C₂H₅ | 3-CF₃,5-CH₂CN |
| A-104 | C₂H₅ | 3-CF₃,5- |
| | | C(CH₃)₂CN |
| A-105 | C₂H₅ | 3-CF₃,5-SO₂CH₃ |
| A-106 | C₂H₅ | 3-CF₃,5-SO₂CF₃ |
| A-107 | C₂H₅ | 3-CF₃,5-OCF₃ |
| A-108 | C₂H₅ | 3-CF₃,5-cC₃H₅ |
| A-109 | C₂H₅ | 3-Cl,5-[(1- |
| | | CN)cC₃H₄] |
| A-110 | C₂H₅ | 3-CF₃,5-[(1- |
| | | CN)cC₃H₄] |
| A-111 | C₂H₅ | 3-CF₃,5-[(2,2- |
| | | Cl₂)-cC₃H₃] |
| A-112 | C₂H₅ | 3-OCF₃,5-cC₃H₅ |
| A-113 | C₂H₅ | 3,5-SO₂CH₃ |
| A-114 | C₂H₅ | 3,5-SO₂CF₃ |
| A-115 | C₂H₅ | 3-Cl,5-OC₆H₅ |
| A-116 | C₂H₅ | 3-CF₃,5-OC₆H₅ |
| A-117 | C₂H₅ | 3-CF₃,5-[O-(4- |
| | | Cl-C₆H₄)] |
| A-118 | CH₂—cC₃H₅ | 3,5-F₂ |
| A-119 | CH₂—cC₃H₅ | 3,5-Cl₂ |
| A-120 | CH₂—cC₃H₅ | 3,5-Br₂ |
| A-121 | CH₂—cC₃H₅ | 3,5-I₂ |
| A-122 | CH₂—cC₃H₅ | 3,5-(CF₃)₂ |
| A-123 | CH₂—cC₃H₅ | 3-Cl,5-F |
| A-124 | CH₂—cC₃H₅ | 3-Cl,5-Br |
| A-125 | CH₂—cC₃H₅ | 3-Cl,5-I |
| A-126 | CH₂—cC₃H₅ | 3-F,5-CN |
| A-127 | CH₂—cC₃H₅ | 3-Cl,5-CN |
| A-128 | CH₂—cC₃H₅ | 3-CF₃,5-CN |
| A-129 | CH₂—cC₃H₅ | 3-F,5-CF₃ |
| A-130 | CH₂—cC₃H₅ | 3-Cl,5-CF₃ |
| A-131 | CH₂—cC₃H₅ | 3-Br,5-CF₃ |
| A-132 | CH₂—cC₃H₅ | 3-I,5-CF₃ |
| A-133 | CH₂—cC₃H₅ | 3-Cl,5-SO₂CH₃ |
| A-134 | CH₂—cC₃H₅ | 3-Cl,5-SO₂CF₃ |
| A-135 | CH₂—cC₃H₅ | 3-Cl,5-cC₃H₅ |
| A-136 | CH₂—cC₃H₅ | 3-Cl,5-[2,2-Cl₂- |
| | | cC₃H₃] |
| A-137 | CH₂—cC₃H₅ | 3-Cl,5-OCF₃ |
| A-138 | CH₂—cC₃H₅ | 3-Br,5-OCF₃ |
| A-139 | CH₂—cC₃H₅ | 3-F,5-cC₃H₅ |
| A-140 | CH₂—cC₃H₅ | 3-Cl,5-CH₂CN |
| A-141 | CH₂—cC₃H₅ | 3-Cl,5- |
| | | C(CH₃)₂CN |
| A-142 | CH₂—cC₃H₅ | 3-CF₃,5-CH₂CN |
| A-143 | CH₂—cC₃H₅ | 3-CF₃,5- |
| | | C(CH₃)₂CN |
| A-144 | CH₂—cC₃H₅ | 3-CF₃,5-SO₂CH₃ |
| A-145 | CH₂—cC₃H₅ | 3-CF₃,5-SO₂CF₃ |
| A-146 | CH₂—cC₃H₅ | 3-CF₃,5-OCF₃ |
| A-147 | CH₂—cC₃H₅ | 3-CF₃,5-cC₃H₅ |
| A-148 | CH₂—cC₃H₅ | 3-Cl,5-[(1- |
| | | CN)cC₃H₄] |
| A-149 | CH₂—cC₃H₅ | 3-CF₃,5-[(1- |
| | | CN)cC₃H₄] |
| A-150 | CH₂—cC₃H₅ | 3-CF₃,5-[(2,2- |
| | | Cl₂)-cC₃H₃] |
| A-151 | CH₂—cC₃H₅ | 3-OCF₃,5-cC₃H₅ |
| A-152 | CH₂—cC₃H₅ | 3,5-SO₂CH₃ |
| A-153 | CH₂—cC₃H₅ | 3,5-SO₂CF₃ |
| A-154 | CH₂—cC₃H₅ | 3-Cl,5-OC₆H₅ |
| A-155 | CH₂—cC₃H₅ | 3-CF₃,5-OC₆H₅ |
| A-156 | CH₂—cC₃H₅ | 3-CF₃,5-[O-(4- |
| | | Cl—C₆H₄)] |
| A-157 | CH₂—cC₅H₉ | 3,5-F₂ |
| A-158 | CH₂—cC₅H₉ | 3,5-Cl₂ |
| A-159 | CH₂—cC₅H₉ | 3,5-Br₂ |
| A-160 | CH₂—cC₅H₉ | 3,5-I₂ |
| A-161 | CH₂—cC₅H₉ | 3,5-(CF₃)₂ |
| A-162 | CH₂—cC₅H₉ | 3-Cl,5-F |
| A-163 | CH₂—cC₅H₉ | 3-Cl,5-Br |
| A-164 | CH₂—cC₅H₉ | 3-Cl,5-I |
| A-165 | CH₂—cC₅H₉ | 3-F,5-CN |
| A-166 | CH₂—cC₅H₉ | 3-Cl,5-CN |
| A-167 | CH₂—cC₅H₉ | 3-CF₃,5-CN |
| A-168 | CH₂—cC₅H₉ | 3-F,5-CF₃ |
| A-169 | CH₂—cC₅H₉ | 3-Cl,5-CF₃ |
| A-170 | CH₂—cC₅H₉ | 3-Br,5-CF₃ |
| A-171 | CH₂—cC₅H₉ | 3-I,5-CF₃ |
| A-172 | CH₂—cC₅H₉ | 3-Cl,5-SO₂CH₃ |
| A-173 | CH₂—cC₅H₉ | 3-Cl,5-SO₂CF₃ |
| A-174 | CH₂—cC₅H₉ | 3-Cl,5-cC₃H₅ |
| A-175 | CH₂—cC₅H₉ | 3-Cl,5-[2,2-Cl₂— |
| | | cC₃H₃] |
| A-176 | CH₂—cC₅H₉ | 3-Cl,5-OCF₃ |
| A-177 | CH₂—cC₅H₉ | 3-Br,5-OCF₃ |
| A-178 | CH₂—cC₅H₉ | 3-F,5-cC₃H₅ |
| A-179 | CH₂—cC₅H₉ | 3-Cl,5-CH₂CN |
| A-180 | CH₂—cC₅H₉ | 3-Cl,5- |
| | | C(CH₃)₂CN |
| A-181 | CH₂—cC₅H₉ | 3-CF₃,5-CH₂CN |
| A-182 | CH₂—cC₅H₉ | 3-CF₃,5- |
| | | C(CH₃)₂CN |
| A-183 | CH₂—cC₅H₉ | 3-CF₃,5-SO₂CH₃ |
| A-184 | CH₂—cC₅H₉ | 3-CF₃,5-SO₂CF₃ |
| A-185 | CH₂—cC₅H₉ | 3-CF₃,5-OCF₃ |
| A-186 | CH₂—cC₅H₉ | 3-CF₃,5-cC₃H₅ |
| A-187 | CH₂—cC₅H₉ | 3-Cl,5-[(1- |
| | | CN)cC₃H₄] |
| A-188 | CH₂—cC₅H₉ | 3-CF₃,5-[(1- |
| | | CN)cC₃H₄] |
| A-189 | CH₂—cC₅H₉ | 3-CF₃,5-[(2,2- |
| | | Cl₂)—cC₃H₃] |
| A-190 | CH₂—cC₅H₉ | 3-OCF₃,5-cC₃H₅ |
| A-191 | CH₂—cC₅H₉ | 3,5-SO₂CH₃ |
| A-192 | CH₂—cC₅H₉ | 3,5-SO₂CF₃ |
| A-193 | CH₂—cC₅H₉ | 3-Cl,5-OC₆H₅ |
| A-194 | CH₂—cC₅H₉ | 3-CF₃,5-OC₆H₅ |
| A-195 | CH₂—cC₅H₉ | 3-CF₃,5-[O-(4- |
| | | Cl—C₆H₄)] |
| A-196 | CH₂CH═CH₂ | 3,5-F₂ |
| A-197 | CH₂CH═CH₂ | 3,5-Cl₂ |
| A-198 | CH₂CH═CH₂ | 3,5-Br₂ |
| A-199 | CH₂CH═CH₂ | 3,5-I₂ |
| A-200 | CH₂CH═CH₂ | 3,5-(CF₃)₂ |
| A-201 | CH₂CH═CH₂ | 3-Cl,5-F |
| A-202 | CH₂CH═CH₂ | 3-Cl,5-Br |
| A-203 | CH₂CH═CH₂ | 3-Cl,5-I |
| A-204 | CH₂CH═CH₂ | 3-F,5-CN |
| A-205 | CH₂CH═CH₂ | 3-Cl,5-CN |
| A-206 | CH₂CH═CH₂ | 3-CF₃,5-CN |
| A-207 | CH₂CH═CH₂ | 3-F,5-CF₃ |
| A-208 | CH₂CH═CH₂ | 3-Cl,5-CF₃ |
| A-209 | CH₂CH═CH₂ | 3-Br,5-CF₃ |
| A-210 | CH₂CH═CH₂ | 3-I,5-CF₃ |
| A-211 | CH₂CH═CH₂ | 3-Cl,5-SO₂CH₃ |
| A-212 | CH₂CH═CH₂ | 3-Cl,5-SO₂CF₃ |
| A-213 | CH₂CH═CH₂ | 3-Cl,5-cC₃H₅ |
| A-214 | CH₂CH═CH₂ | 3-Cl,5-[2,2-Cl₂— |
| | | cC₃H₃] |
| A-215 | CH₂CH═CH₂ | 3-Cl,5-OCF₃ |
| A-216 | CH₂CH═CH₂ | 3-Br,5-OCF₃ |
| A-217 | CH₂CH═CH₂ | 3-F,5-cC₃H₅ |
| A-218 | CH₂CH═CH₂ | 3-Cl,5-CH₂CN |
| A-219 | CH₂CH═CH₂ | 3-Cl,5- |
| | | C(CH₃)₂CN |
| A-220 | CH₂CH═CH₂ | 3-CF₃,5-CH₂CN |
| A-221 | CH₂CH═CH₂ | 3-CF₃,5- |
| | | C(CH₃)₂CN |
| A-222 | CH₂CH═CH₂ | 3-CF₃,5-SO₂CH₃ |
| A-223 | CH₂CH═CH₂ | 3-CF₃,5-SO₂CF₃ |
| A-224 | CH₂CH═CH₂ | 3-CF₃,5-OCF₃ |
| A-225 | CH₂CH═CH₂ | 3-CF₃,5-cC₃H₅ |
| A-226 | CH₂CH═CH₂ | 3-Cl,5-[(1- |
| | | CN)cC₃H₄] |
| A-227 | CH₂CH═CH₂ | 3-CF₃,5-[(1- |
| | | CN)cC₃H₄] |
| A-228 | CH₂CH═CH₂ | 3-CF₃,5-[(2,2- |
| | | Cl₂)—cC₃H₃] |
| A-229 | CH₂CH═CH₂ | 3-OCF₃,5-cC₃H₅ |
| A-230 | CH₂CH═CH₂ | 3,5-SO₂CH₃ |
| A-231 | CH₂CH═CH₂ | 3,5-SO₂CF₃ |
| A-232 | CH₂CH═CH₂ | 3-Cl,5-OC₆H₅ |
| A-233 | CH₂CH═CH₂ | 3-CF₃,5-OC₆H₅ |
| A-234 | CH₂CH═CH₂ | 3-CF₃,5-[O-(4- |
| | | Cl—C₆H₄)] |
| A-235 | CH₂C≡CH | 3,5-F₂ |
| A-236 | CH₂C≡CH | 3,5-Cl₂ |
| A-237 | CH₂C≡CH | 3,5-Br₂ |
| A-238 | CH₂C≡CH | 3,5-I₂ |
| A-239 | CH₂C≡CH | 3,5-(CF₃)₂ |
| A-240 | CH₂C≡CH | 3-Cl,5-F |
| A-241 | CH₂C≡CH | 3-Cl,5-Br |
| A-242 | CH₂C≡CH | 3-Cl,5-I |
| A-243 | CH₂C≡CH | 3-F,5-CN |
| A-244 | CH₂C≡CH | 3-Cl,5-CN |
| A-245 | CH₂C≡CH | 3-CF₃,5-CN |
| A-246 | CH₂C≡CH | 3-F,5-CF₃ |
| A-247 | CH₂C≡CH | 3-Cl,5-CF₃ |
| A-248 | CH₂C≡CH | 3-Br,5-CF₃ |
| A-249 | CH₂C≡CH | 3-I,5-CF₃ |
| A-250 | CH₂C≡CH | 3-Cl,5-SO₂CH₃ |
| A-251 | CH₂C≡CH | 3-Cl,5-SO₂CF₃ |
| A-252 | CH₂C≡CH | 3-Cl,5-cC₃H₅ |
| A-253 | CH₂C≡CH | 3-Cl,5-[2,2-Cl₂— |
| | | cC₃H₃] |
| A-254 | CH₂C≡CH | 3-Cl,5-OCF₃ |
| A-255 | CH₂C≡CH | 3-Br,5-OCF₃ |
| A-256 | CH₂C≡CH | 3-F,5-cC₃H₅ |
| A-257 | CH₂C≡CH | 3-Cl,5-CH₂CN |
| A-258 | CH₂C≡CH | 3-Cl,5- |
| | | C(CH₃)₂CN |
| A-259 | CH₂C≡CH | 3-CF₃,5-CH₂CN |
| A-260 | CH₂C≡CH | 3-CF₃,5- |
| | | C(CH₃)₂CN |
| A-261 | CH₂C≡CH | 3-CF₃,5-SO₂CH₃ |
| A-262 | CH₂C≡CH | 3-CF₃,5-SO₂CF₃ |
| A-263 | CH₂C≡CH | 3-CF₃,5-OCF₃ |
| A-264 | CH₂C≡CH | 3-CF₃,5-cC₃H₅ |
| A-265 | CH₂C≡CH | 3-Cl,5-[(1- |
| | | CN)cC₃H₄] |
| A-266 | CH₂C≡CH | 3-CF₃,5-[(1- |
| | | CN)cC₃H₄] |
| A-267 | CH₂C≡CH | 3-CF₃,5-[(2,2- |
| | | Cl₂)—cC₃H₃] |
| A-268 | CH₂C≡CH | 3-OCF₃,5-cC₃H₅ |
| A-269 | CH₂C≡CH | 3,5-SO₂CH₃ |
| A-270 | CH₂C≡CH | 3,5-SO₂CF₃ |
| A-271 | CH₂C≡CH | 3-Cl,5-OC₆H₅ |
| A-272 | CH₂C≡CH | 3-CF₃,5-OC₆H₅ |
| A-273 | CH₂C≡CH | 3-CF₃,5-[O—(4- |
| | | Cl—C₆H₄)] |

The term “compound(s) of the invention” refers to compound(s) of formula I, or “compound(s) I”, and includes their salts, tautomers, stereoisomers, and N-oxides.

The invention also relates to agrochemical compositions comprising an auxiliary and at least one compound I.

An agrochemical composition comprises a pesticidally effective amount of a compound I.

An agrochemical composition comprises a pesticidally effective amount of a compound I.

The compounds I can be converted into customary types of agro-chemical compositions, e.g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials e.g. seeds (e.g. GF). These and further compositions types are defined in the “Catalogue of pesticide formulation types and international coding system”, Technical Monograph No. 2, 6th Ed. May 2008, CropLife International.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents. Suitable solid carriers or fillers are mineral earths.

Suitable surfactants are surface-active compounds, e.g. anionic, cationic, nonionic, and amphoteric surfactants, block polymers, polyelectrolytes. Such surfactants can be used as emulsifier, dispersant, solubilizer, wetter, penetration enhancer, protective colloid, or adjuvant. Surfactants are listed in McCutcheon's, Vol. 1: Emulsifiers & Detergents, McCutcheon's Directories, Glen Rock, USA, 2008 (International or North American Ed.). Suitable anionic surfactants are alkali, alkaline earth, or ammonium salts of sulfonates, sulfates, phosphates, carboxylates. Suitable nonionic surfactants are alkoxylates, N-subsituted fatty acid amides, amine oxides, esters, sugar-based surfactants, polymeric surfactants. Suitable cationic surfactants are quaternary surfactants.

The agrochemical compositions generally comprise between 0.01 and 95%, preferably between 0.1 and 90%, and most preferably between 0.5 and 75%, by weight of active substance.

The active substances are employed in a purity of from 90% to 100%, preferably from 95% to 100%.

Various types of oils, wetters, adjuvants, or fertilizer may be added to the active substances or the compositions comprising them as premix or, if appropriate not until immediately prior to use (tank mix). These agents can be admixed with the compositions according to the invention in a weight ratio of 1:100 to 100:1.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters of the ready-to-use spray liquor are applied per hectare of agricultural useful area.

The compounds I are suitable for use in protecting crops, plants, plant propagation materials, e.g. seeds, or soil or water, in which the plants are growing, from attack or infestation by animal pests. Therefore, the invention also relates to a plant protection method, which comprises contacting crops, plants, plant propagation materials, e.g. seeds, or soil or water, in which the plants are growing, to be protected from attack or infestation by animal pests, with a pesticidally effective amount of a compound I.

The compounds I are also suitable for use in combating or controlling animal pests. Therefore, the invention also relates to a method of combating or controlling animal pests, which comprises contacting the animal pests, their habitat, breeding ground, or food supply, or the crops, plants, plant propagation materials, e.g. seeds, or soil, or the area, material or environment in which the animal pests are growing or may grow, with a pesticidally effective amount of a compound I.

The compounds I are effective through both contact and ingestion to any and all developmental stages, such as egg, larva, pupa, and adult.

The compounds I can be applied as such or in form of compositions comprising them.

The application can be carried out both before and after the infestation of the crops, plants, plant propagation materials by the pests.

The term “contacting” includes both direct contact (applying the compounds/compositions directly on the animal pest or plant) and indirect contact (applying the compounds/compositions to the locus).

The term “animal pest” includes arthropods, gastropods, and nematodes. Preferred animal pests according to the invention are arthropods, preferably insects and arachnids, in particular insects.

The term “plant” includes cereals, e.g. durum and other wheat, rye, barley, triticale, oats, rice, or maize (fodder maize and sugar maize/sweet and field corn); beet, e.g. sugar beet, or fodder beet; fruits, e.g. pomes, stone fruits, or soft fruits, e.g. apples, pears, plums, peaches, nectarines, almonds, cherries, papayas, strawberries, raspberries, blackberries or gooseberries; leguminous plants, e.g. beans, lentils, peas, alfalfa, or soybeans; oil plants, e.g. rapeseed (oilseed rape), turnip rape, mustard, olives, sunflowers, coconut, cocoa beans, castor oil plants, oil palms, ground nuts, or soybeans; cucurbits, e.g. squashes, pumpkins, cucumber or melons; fiber plants, e.g. cotton, flax, hemp, or jute; citrus fruit, e.g. oranges, lemons, grapefruits or mandarins; vegetables, e.g. eggplant, spinach, lettuce (e.g. iceberg lettuce), chicory, cabbage, asparagus, cabbages, carrots, onions, garlic, leeks, tomatoes, potatoes, cucurbits or sweet peppers; lauraceous plants, e.g. avocados, cinnamon, or camphor; energy and raw material plants, e.g. corn, soybean, rapeseed, sugar cane or oil palm; tobacco; nuts, e.g. walnuts; pistachios; coffee; tea; bananas; vines; hop; sweet leaf (Stevia); natural rubber plants or ornamental and forestry plants, shrubs, broad-leaved trees or evergreens, eucalyptus; turf; lawn; grass. Preferred plants include potatoes, sugar beets, tobacco, wheat, rye, barley, oats, rice, corn, cotton, soybeans, rapeseed, legumes, sunflowers, coffee, or sugar cane; fruits; vines; ornamentals; or vegetables, e.g. cucumbers, tomatoes, beans or squashes.

The term “seed” embraces seeds and plant propagules including true seeds, seed pieces, suckers, corms, bulbs, fruit, tubers, grains, cuttings, cut shoots, and means preferably true seeds.

“Pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions e.g. desired pesticidal effect and duration, weather, target species, locus, mode of application.

For use in treating crop plants, e.g. by foliar application, the rate of application of the active ingredients of this invention may be in the range of 0.0001 g to 4000 g per hectare, e.g. from 1 g to 2 kg per hectare or from 1 g to 750 g per hectare, desirably from 1 g to 100 g per hectare.

The compounds I are also suitable for use against non-crop insect pests. For use against said non-crop pests, compounds I can be used as bait composition, gel, general insect spray, aerosol, as ultra-low volume application and bed net (impregnated or surface applied).

The term “non-crop insect pest” refers to pests, which are particularly relevant for non-crop targets, e.g. ants, termites, wasps, flies, ticks, mosquitoes, bed bugs, crickets, or cockroaches, such as: Aedes aegypti, Musca domestica, Tribolium spp.; termites such as Reticulitermes flavipes, Coptotermes formosanus; roaches such as Blatella germanica, Periplaneta Americana; ants such as Solenopsis invicta, Linepithema humile, and Camponotus pennsylvanicus.

The bait can be a liquid, a solid or a semisolid preparation (e.g. a gel). For use in bait compositions, the typical content of active ingredient is from 0.001 wt % to 15 wt %, desirably from 0.001 wt % to 5 wt % of active compound.

The compounds I and its compositions can be used for protecting wooden materials such as trees, board fences, sleepers, frames, artistic artifacts, etc. and buildings, but also construction materials, furniture, leathers, fibers, vinyl articles, electric wires and cables etc. from ants, termites and/or wood or textile destroying beetles, and for controlling ants and termites from doing harm to crops or human beings (e.g. when the pests invade into houses and public facilities or nest in yards, orchards or parks).

Customary application rates in the protection of materials are, e.g., from 0.001 g to 2000 g or from 0.01 g to 1000 g of active compound per m² treated material, desirably from 0.1 g to 50 g per m².

Insecticidal compositions for use in the impregnation of materials typically contain from 0.001 to 95 wt %, preferably from 0.1 to 45 wt %, and more preferably from 1 to 25 wt % of at least one repellent and/or insecticide.

The compounds of the invention are especially suitable for efficiently combating animal pests e.g. arthropods, and nematodes including:
insects from the sub-order of Auchenorrhyncha, e.g. Amrasca biguttula, Empoasca spp., Nephotettix virescens, Sogatella furcifera, Mahanarva spp., Laodelphax striatellus, Nilaparvata lugens, Diaphorina citri; Lepidoptera, e.g. Helicoverpa spp., Heliothis virescens, Lobesia botrana, Ostrinia nubilalis, Plu-tella xylostella, Pseudoplusia includens, Scirpophaga incertulas, Spodoptera spp., Trichoplusia ni, Tuta absoluta, Cnaphalocrocis medialis, Cydia pomonella, Chilo suppressalis, Anticarsia gemmatalis, Agrotis ipsilon, Chrysodeixis includens;
True bugs, e.g. Lygus spp., Stink bugs such as Euschistus spp., Halyomorpha halys, Nezara viridula, Piezodorus guildinii, Dichelops furcatus;
Thrips, e.g. Frankliniella spp., Thrips spp., Dichromothrips corbettii;
Aphids, e.g. Acyrthosiphon pisum, Aphis spp., Myzus persicae, Rhopalosiphum spp., Schizaphis graminum, Megoura viciae;
Whiteflies, e.g. Trialeurodes vaporariorum, Bemisia spp.;
Coleoptera, e.g. Phyllotreta spp., Melanotus spp., Meligethes aeneus, Leptinotarsa decimlineata, Ceutorhynchus spp., Diabrotica spp., Anthonomus grandis, Atomaria linearia, Agriotes spp., Epilachna spp.;
Flies, e.g. Delia spp., Ceratitis capitate, Bactrocera spp., Liriomyza spp.;
Mosquitoes (Diptera), e.g. Aedes aegypti, A. albopictus, A. vexans, Anastrepha ludens, Anopheles maculipennis, A. crucians, A. albimanus, A. gambiae, A. freeborni, A. leucosphyrus, A. minimus, A. quadrimaculatus;
Coccoidea, e.g. Aonidiella aurantia, Ferrisia virgate;
Anthropods of class Arachnida (Mites), e.g. Penthaleus major, Tetranychus spp.;
Nematodes, e.g. Heterodera glycines, Meloidogyne sp., Pratylenchus spp., Caenorhabditis elegans.

The compounds I are suitable for use in treating or protecting animals against infestation or infection by parasites. Therefore, the invention also relates to the use of a compound of the invention for the manufacture of a medicament for the treatment or protection of animals against infestation or infection by parasites. Furthermore, the invention relates to a method of treating or protecting animals against infestation and infection by parasites, which comprises orally, topically or parenterally administering or applying to the animals a parasiticidally effective amount of a compound I.

The invention also relates to the non-therapeutic use of compounds of the invention for treating or protecting animals against infestation and infection by parasites. Moreover, the invention relates to a non-therapeutic method of treating or protecting animals against infestation and infection by parasites, which comprises applying to a locus a parasiticidally effective amount of a compound I.

The compounds of the invention are further suitable for use in combating or controlling parasites in and on animals. Furthermore, the invention relates to a method of combating or controlling parasites in and on animals, which comprises contacting the parasites with a parasitically effective amount of a compound I.

The invention also relates to the non-therapeutic use of compounds I for controlling or combating parasites. Moreover, the invention relates to a non-therapeutic method of combating or controlling parasites, which comprises applying to a locus a parasiticidally effective amount of a compound I.

The compounds I can be effective through both contact (via soil, glass, wall, bed net, carpet, blankets, or animal parts) and ingestion (e.g. baits). Furthermore, the compounds I can be applied to any and all developmental stages.

The compounds I can be applied as such or in form of compositions comprising them.

The term “locus” means the habitat, food supply, breeding ground, area, material or environment in which a parasite is growing or may grow outside of the animal.

As used herein, the term “parasites” includes endo- and ectoparasites. In some embodiments of the invention, endoparasites can be preferred. In other embodiments, ectoparasites can be preferred. Infestations in warm-blooded animals and fish include lice, biting lice, ticks, nasal bots, keds, biting flies, muscoid flies, flies, myiasitic fly larvae, chiggers, gnats, mosquitoes and fleas.

The compounds of the invention are especially useful for combating the following parasites: Cimex lectularius, Rhipicephalus sanguineus, and Ctenocephalides felis.

As used herein, the term “animal” includes warm-blooded animals (including humans) and fish.

Preferred are mammals, such as cattle, sheep, swine, camels, deer, horses, pigs, poultry, rabbits, goats, dogs and cats, water buffalo, donkeys, fallow deer and reindeer, and also in furbearing animals such as mink, chinchilla and raccoon, birds such as hens, geese, turkeys and ducks and fish such as fresh- and salt-water fish such as trout, carp and eels. Particularly preferred are domestic animals, such as dogs or cats.

The compounds I may be applied in total amounts of 0.5 mg/kg to 100 mg/kg per day, preferably 1 mg/kg to 50 mg/kg per day.

For oral administration to warm-blooded animals, the compounds I may be formulated as animal feeds, animal feed premixes, animal feed concentrates, pills, solutions, pastes, suspensions, drenches, gels, tablets, boluses and capsules. For oral administration, the dosage form chosen should provide the animal with 0.01 mg/kg to 100 mg/kg of animal body weight per day of the compounds I, preferably with 0.5 mg/kg to 100 mg/kg of animal body weight per day.

Alternatively, the compounds I may be administered to animals parenterally, e.g., by intraruminal, intramuscular, intravenous or subcutaneous injection. The compounds I may be dispersed or dissolved in a physiologically acceptable carrier for subcutaneous injection. Alternatively, the compounds I may be formulated into an implant for subcutaneous administration. In addition, the compounds I may be transdermally administered to animals. For parenteral administration, the dosage form chosen should provide the animal with 0.01 mg/kg to 100 mg/kg of animal body weight per day of the compounds I.

The compounds I may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the compounds I. In addition, the compounds I may be formulated as ear tags for animals, particularly quadrupeds e.g. cattle and sheep.

Oral solutions are administered directly.

Solutions for use on the skin are trickled on, spread on, rubbed in, sprinkled on or sprayed on.

Gels are applied to or spread on the skin or introduced into body cavities.

Pour-on formulations are poured or sprayed onto limited areas of the skin, the active compound penetrating the skin and acting systemically. Pour-on formulations are prepared by dissolving, suspending, or emulsifying the active compound in suitable skin-compatible solvents or solvent mixtures.

Emulsions can be administered orally, dermally or as injections.

Suspensions can be administered orally or topically/dermally.

Semi-solid preparations can be administered orally or topically/dermally.

For the production of solid preparations, the active compound is mixed with suitable excipients, if appropriate with addition of auxiliaries, and brought into the desired form.

The compositions which can be used in the invention can comprise generally from about 0.001 to 95% of the compound I.

Ready-to-use preparations contain the compounds acting against parasites, preferably ectoparasites, in concentrations of 10 ppm to 80% by weight, preferably from 0.1 to 65% by weight, more preferably from 1 to 50% by weight, most preferably from 5 to 40% by weight.

Preparations which are diluted before use contain the compounds acting against ectoparasites in concentrations of 0.5 to 90% by weight, preferably of 1 to 50% by weight.

Furthermore, the preparations comprise the compounds of formula I against endoparasites in concentrations of 10 ppm to 2% by weight, preferably of 0.05 to 0.9% by weight, very particularly preferably of 0.005 to 0.25% by weight.

Solid formulations which release compounds of the invention may be applied in total amounts of 10 mg/kg to 300 mg/kg, preferably 20 mg/kg to 200 mg/kg, most preferably 25 mg/kg to 160 mg/kg body weight of the treated animal in the course of three weeks.

### A. PREPARATION EXAMPLES

The compounds were characterized by melting point determination, by NMR spectroscopy or by the mass-to-charge ratio ([m/z]) and retention time (RT; [min.]), as determined by mass spectrometry (MS) coupled with HPLC analysis (HPLC-MS=high performance liquid chromatography-coupled mass spectrometry) or LC analysis (LC-MS=liquid chromatography-coupled mass spectrometry).
Method A: HPLC: Shimadzu Nexera UHPLC+Shimadzu LCMS-2020, ESI; Column: Phenomenex Kinetex 1.7 μm XB—C18 100A, 2.1×50 mm; Mobile phase: A: water+0.1% TFA; B: ACN; Temperature: 60° C.; Gradient: 5% B to 100% B in 1.5 min; 100% B 0.25 min; Flow: 0.8 mL/min to 1.0 mL/min in 1.51 min; MS: ESI positive; Mass range (m/z): 100-700.
Method B: LC: Shimadzu LC-30AD, ESI; Column: Kinetex EVO C18.5 μm 2.1×30 mm; Mobile phase: A: water+0.04% TFA; B: ACN+0.02% TFA; Temperature: 40° C.; Gradient: 5% B to 100% B in 2.5 min; 100% B to 5% B in 0.02 min; 5% B for 0.5 min; Flow: 0.8 mL/min; MS: ESI positive; Mass range: 100-2000.
Method C: HPLC/MS: Agilent 1260 HPLC MSD: 6125B single quadrupole MSD; Column: Luna C18 2.0×50 mm 5 μm; Mobile phase: A: 0.04% TFA in water; B: 0.02% TFA in ACN; Temperature: 40° C.; Gradient: 5% B for 0.4 min; 5% B to 95% B in 2.6 min; 95% B for 1 min; 95% B to 5% B in 0.01 min; 5% B for 0.5 min; Flow: 1.0 mL/min; MS: ES-API positive; Mass range: 100-1000.

### Example 1: Preparation of 3-bromo-N-[1-[3-[1-(2,2,2-trifluoroethyl)-1,2,4-triazol-3-yl]pyrazin-2-yl]ethyl]-5-(trifluoromethyl)benzamide[I1-1]

### Step 1: Synthesis of (N)-3-chloro-N-(dimethylaminomethylene)pyrazine-2-carboxamide

To a solution of 3-chloropyrazine-2-carboxamide (3 g, 0.0191 mol) in DCM (30 mL) was added DMF-dimethyl acetal (4.5 g, 0.0382 mol) at 20° C. The mixture was stirred at 50° C. for 2 h, LCMS showed the reaction was completed. The reaction mixture was concentrated to give the title compound (3 g, yield: crude) as a white solid, which was used for the next step directly.

### Step 2: Synthesis of 2-chloro-3-(1H-1,2,4-triazol-3-yl)pyrazine

To a solution of (N)-3-chloro-N-(dimethylaminomethylene)pyrazine-2-carboxamide (3 g, 0.0141 mol) in 1,4-dioxane (30 mL) was added NH₂NH₂×H₂O (1.4 g, 0.0283 mol) and AcOH (30 mL) at 20° C. The mixture was stirred at 80° C. for 2 h, LCMS showed the reaction was completed. The reaction mixture was concentrated to give crude 2-chloro-3-(1H-1,2,4-triazol-3-yl)pyrazine (3 g, crude) as a yellow solid, which was directly used for the next step. A small portion was crystallized for characterization.

¹H-NMR (400 MHz, DMSO-d6): δ=8.64 (m, 2H), 8.80 (d, 1H).

### Step 3: Synthesis of 2-chloro-3-[1-(2,2,2-trifluoroethyl)-1,2,4-triazol-3-yl]pyrazine

To a mixture of 2-chloro-3-(1H-1,2,4-triazol-3-yl)pyrazine (1.6 g, 0.0088 mol) in MeCN (80 mL) was added 2,2,2-trifluoroethyl trifluoromethanesulfonate (2.45 g, 0.0106 mol) and K₂CO₃ (2.276 g, 0.0176 mol) at 20° C. The reaction mixture was stirred at 25° C. for 12 h. LCMS showed 70% of the product. The reaction mixture was quenched with H₂O (20 mL), extracted with EtOAc (3×80 mL), the separated organic layers were washed with brine (20 mL), dried over Na₂SO₄ and concentrated. The residue was purified by silica gel column (PE:EtOAc=100:0 gradient to 20:80) to give 2-chloro-3-[1-(2,2,2-trifluoroethyl)-1,2,4-triazol-3-yl]pyrazine (0.600 g, yield: 26%).

¹H NMR (400 MHz, CDCl3): 5=5.24 (q, 2H), 8.17 (s, 1H), 8.57 (d, 1H), 8.64 (d, 1H).

### Step 4: Synthesis of 2-(1-ethoxyvinyl)-3-[1-(2,2,2-trifluoroethyl)-1,2,4-triazol-3-yl]pyrazine

To a solution of 2-chloro-3-[1-(2,2,2-trifluoroethyl)-1,2,4-triazol-3-yl]pyrazine (3.8 g, 0.0144 mol) in toluene (100 mL) was added tributyl(1-ethoxyvinyl)stannane (5.21 g, 0.0144 mol) and Pd(PPh₃)₂Cl₂ (1 g) at 25° C. The reaction mixture was stirred for 12 h at 110° C. LCMS showed the reaction was completed. After the reaction mixture was cooled down to room temperature, saturated aqueous KF (150 mL) was added to the reaction mixture and stirred for another 30 min. The mixture was filtered through a celite pad and the filtrate was extracted with EtOAc (3×50 mL). The separated organic phase was washed with brine (60 mL), dried over Na₂SO₄ and concentrated. The residue was purified by silica gel column (PE:EtOAc=100:0 gradient to 10:90) to give the title compound (3.1 g, yield: 72%).

¹H NMR (400 MHz, CDCl₃): δ=1.01 (t, 3H), 3.78 (q, 2H), 4.49 (d, 1H), 4.84-4.88 (m, 1H), 4.89-4.94 (m, 2H), 8.30 (s, 1H), 8.61 (m, 2H).

### Step 5: Synthesis of 1-[3-[1-(2,2,2-trifluoroethyl)-1,2,4-triazol-3-yl]pyrazin-2-yl]ethanone

To a solution of 2-(1-ethoxyvinyl)-3-[1-(2,2,2-trifluoroethyl)-1,2,4-triazol-3-yl]pyrazine (3.1 g, 0.0103 mol) in THF (30 mL) was added aqueous HCl (2M, 30 mL) dropwise at 25° C. The mixture was stirred for 16 h at 25° C. LCMS showed the reaction was completed. The reaction mixture was diluted with H₂O (50 mL), extracted with EtOAc (3×20 mL). The separated organic phase was washed with brine (40 mL), dried over Na₂SO₄, and concentrated to give the title compound (2.9 g, crude).

¹H-NMR (400 MHz, CDCl₃): δ=2.70 (s, 3H), 4.97 (q, 2H), 8.69 (s, 2H), 8.80 (br s, 1H).

### Step 6: Synthesis of 1-[3-[1-(2,2,2-trifluoroethyl)-1,2,4-triazol-3-yl]pyrazin-2-yl]ethanamine

To a solution of 1-[3-[1-(2,2,2-trifluoroethyl)-1,2,4-triazol-3-yl]pyrazin-2-yl]ethanone (1.3 g, 4.79 mmol) in MeOH (50 mL) was added NH₄OAc (3.69 g, 48 mmol), NaBH₃CN (602 mg, 9.59 mmol) and the reaction mixture was stirred for 16 h at 20° C. TLC (DCM:MeOH=10:1) showed the reaction was completed. The reaction mixture was concentrated and quenched with H₂O (50 mL), the pH was adjusted to pH-10 with aq. NaOH. The mixture was extracted with DCM/iPrOH (3/1, 3×20 mL), the combined organic phases were dried over Na₂SO₄ and concentrated to give crude 1-[3-[1-(2,2,2-trifluoroethyl)-1,2,4-triazol-3-yl]pyrazin-2-yl]ethanamine (500 mg, crude), which was used in the next step without further purification.

### Step 7: Synthesis of 3-bromo-N-[1-[3-[1-(2,2,2-trifluoroethyl)-1,2,4-triazol-3-yl]pyrazin-2-yl]ethyl]-5-(trifluoromethyl)benzamide

To a solution of 3-bromo-5-(trifluoromethyl)benzoic acid (0.494 g, 0.00184 mol) in MeCN (10 mL) was added N,N,N′,N′-tetramethylchloroformamidinium hexafluorophosphate (0.773 g, 0.00276 mol), N-methylimidazole (0.453 g, 0.0055 mol) and 1-[3-[1-(2,2,2-trifluoroethyl)-1,2,4-triazol-3-yl]pyrazin-2-yl]ethanamine (0.5 g, 0.00184 mol) at 20° C. The mixture was stirred at 20° C. for 1 h. LCMS showed the reaction was completed. The reaction mixture was quenched with H₂O (10 mL), extracted with EtOAc (3×30 mL): The combined organic layers were washed with brine (10 mL), dried over Na₂SO₄ and concentrated. The residue was purified by Prep-HPLC (NHaHCO₃) to give the title compound (11-1, 0.375 g, yield: 39%) as a white solid.

¹H-NMR (400 MHz, CDCl₃): δ=1.63 (d, 3H), 4.98 (m, 2H), 5.23 (quin, 1H), 7.84 (br d, 1H), 7.90 (s, 1H), 8.02 (s, 1H), 8.15 (s, 1H), 8.43 (s, 1H), 8.67 (d, 1H), 8.74 (d, 1H).

LCMS (Method B): (Desired Mass: m/z=523; Observed Mass: m/z=523.525).

### Example 2: Preparation of 2,6-dichloro-N-[1-[3-(1-phenyl-1,2,4-triazol-3-yl)pyrazin-2-yl]ethyl]pyridine-4-carboxamide [I2-1]

### Step 1: Preparation of 2-chloro-3-[1-[(4-methoxyphenyl)methyl]-1,2,4-triazol-3-yl]pyrazine

To a solution of 3-chloro-N-(dimethylaminomethylene)pyrazine-2-carboxamide (84 g, 376.2 mmol) in 1,4-dioxan (840 ml) was added [(4-methoxyphenyl)methylamino]ammonium chloride (142 g, 752.4 mmol) at 15° C. and stirred for 30 min. Acetic acid (840 mL) was added and the mixture was stirred at 15° C. for further 2 h. Then the reaction mixture was heated to 80° C. and stirred for 16 h. LC-MS showed the reaction was completed. The reaction was quenched with water (600 mL). The resulting mixture was extracted with EtOAc (3×1 L). The combined organic phase was washed with brine (300 mL), dried over Na₂SO₄, filtered and concentrated under reduced pressure. The residue was purified by column (EtOAc) to give a mixture of 2-chloro-3-[1-[(4-methoxyphenyl)methyl]-1,2,4-triazol-3-yl]pyrazine and 2-chloro-3-[2-[(4-methoxyphenyl)methyl]-1,2,4-triazol-3-yl]pyrazine (66 g crude) as a yellow oil.

¹H-NMR (400 MHz, CDCl₃): δ=8.62 (d, J=2.38 Hz, 1H), 8.48 (d, J=2.38 Hz, 1H), 8.08 (s, 1H), 7.06 (d, J=8.66 Hz, 2H), 6.75-6.80 (m, 2H), 5.45 (s, 2H), 3.75 (s, 3H).

### Step 2: Preparation of 1-[3-[1-[(4-methoxyphenyl)methyl]-1,2,4-triazol-3-yl]pyrazin-2-yl]ethanone

To a solution of a mixture of 2-chloro-3-[1-[(4-methoxyphenyl)methyl]-1,2,4-triazol-3-yl]pyrazine and 2-chloro-3-[2-[(4-methoxyphenyl)methyl]-1,2,4-triazol-3-yl]pyrazine (66 g, 218.7 mmol) in DMF was added Pd(PPh₃)₂Cl₂ (15.3 g, 21.87 mmol) and tributyl(1-ethoxyvinyl)stannane (18.5 g, 328.1 mmol) at 15° C. The reaction mixture was heated to 100° C. and stirred for 16 h. TCL (EtOAc) showed that the reaction was completed. The reaction mixture was poured into KF (aq. sat, 1 L) and stirred for 2.5 h. The mixture was filtered and the filtrate was extracted with EtOAc (3×1 L). The combined organic phase was dried over Na₂SO₄, filtered and concentrated under reduced pressure. The residue was purified by column (EtOAc) to give a mixture of 1-[3-[1-[(4-methoxyphenyl)methyl]-1,2,4-triazol-3-yl]pyrazin-2-yl]ethenone and 1-[3-[2-[(4-methoxyphenyl)methyl]-1,2,4-triazol-3-yl]pyrazin-2-yl]ethanone (57 g crude) as a yellow solid.

¹H-NMR (400 MHz, CDCl₃): δ=8.75 (d, J=2.38, 1H), 8.66 (d, J=2.50 Hz, 1H), 7.96 (s, 1H), 7.18-7.24 (m, 2H), 6.78-6.86 (m, 2H), 5.60 (s, 2H), 3.77 (s, 3H), 2.72 (s, 3H).

### Step 3: Preparation of 1-[3-(1H-1,2,4-triazol-3-yl)pyrazin-2-yl]ethanone

A solution of 1-[3-[1-[(4-methoxyphenyl)methyl]-1,2,4-triazol-3-yl]pyrazin-2-yl]ethanone and 1-[3-[2-[(4-methoxyphenyl)methyl]-1,2,4-triazol-3-yl]pyrazin-2-yl]ethanone (5.7 g, 18.43 mmol) in TFA (57 ml) was stirred at 80° C. for 16 h. LC-MS showed the reaction was completed. The mixture was concentrated under reduced pressure and purified by column (EtOAc:EtOH=3:1) to give 1-[3-(1H-1,2,4-triazol-3-yl)pyrazin-2-yl]ethanone (3.4 g, crude) as a light yellow solid.

¹H-NMR (400 MHz, CDCl₃): δ=8.81 (d, J=2.50 Hz, 1H), 8.71 (d, J=2.50 Hz, 1H), 8.46 (br s, 1H), 2.69 (s, 3H).

### Step 4: Preparation of 1-[3-[1-(4-nitrophenyl)-1,2,4-triazol-3-yl]pyrazin-2-yl]ethenone

To a solution of 1-[3-(1H-1,2,4-triazol-3-yl)pyrazin-2-yl]ethanone (3 g, 15.86 mmol) in DMF was added NaH (571 mg, 23.79 mmol) at 0° C. A solution of 1-fluoro-4-nitro-benzene (2.91 g, 20.62 mmol) in DMF (10 mL) was added. The reaction mixture was stirred at 15° C. for 16 h. TLC (EtOAc:EtOH=3:1) showed the reaction was completed. The mixture was quenched with water (120 mL) and extracted with EtOAc (3×80 mL). The combined organic phase was washed with brine (3×80 ml), dried over Na₂SO₄, filtered and concentrated under reduced pressure. The residue was purified by column (EtOAc:EtOH=3:1) to give 1-[3-[1-(4-nitrophenyl)-1,2,4-triazol-3-yl]pyrazin-2-yl]ethanone (4.5 g, 91.5% yield) as a yellow solid.

¹H-NMR (400 MHz, CDCl₃): δ=8.83 (d, J=2.38 Hz, 1H), 8.79 (s, 1H), 8.69 (d, J=2.38 Hz, 1H), 8.40-8.45 (m, 2H), 7.96-8.00 (m, 2H), 2.80 (s, 3H).

### Step 5: Preparation of 1-[3-[1-(4-aminophenyl)-1,2,4-triazol-3-yl]pyrazin-2-yl]ethanone

To a solution of 1-[3-[1-(4-nitrophenyl)-1,2,4-triazol-3-yl]pyrazin-2-yl]ethanone (4.4 g, 14.18 mmol) in MeOH (45 ml) was added SnC1₂ (8.067 g, 42.54 mmol) at 15° C. The reaction mixture was heated to 80° C. and stirred for 3 h. LC-MS showed the reaction was completed. The mixture was concentrated and then quenched with water (20 ml). The pH was adjusted to pH-9 with NaOH (aq. 2N) and then extracted with EtOAc (3×20 mL). The combined organic phase was washed with brine (20 mL), dried over Na₂SO₄, filtered and concentrated under reduced pressure. The residue was purified by column (EtOAc:EtOH=3:1) to give 1-[3-[1-(4-aminophenyl)-1,2,4-triazol-3-yl]pyrazin-2-yl]ethanone (750 mg, 19% yield) as a yellow solid.

¹H-NMR (400 MHz, DMSO-d₆): δ=9.15 (s, 1H), 8.89 (d, J=2.50 Hz, 1H), 8.76 (d, J=2.50 Hz, 1H), 7.46-7.54 (m, 2H), 6.66-6.72 (m, 2H), 5.49 (s, 2H), 2.64 (s, 3H).

### Step 6: Preparation of 1-[3-(1-phenyl-1,2,4-triazol-3-yl)pyrazin-2-yl]ethanone

To a solution of 1-[3-[1-(4-aminophenyl)-1,2,4-triazol-3-yl]pyrazin-2-yl]ethanone (720 mg, 2.57 mmol) in DMF (8 mL) was added NaNO₂ (355 mg, 5.14 mmol) and Et₂O—BF₃ (725 mg, 5.14 mmol) at 15° C. The reaction mixture was heated to 50° C. and stirred for 16 h. LC-MS showed reaction was completed. The mixture was quenched with water (30 mL) and extracted with EtOAc (3×20 mL). The combined organic phase was washed with brine (3×20 mL), dried over Na2SO4, filtered and concentrated under reduced pressure. The residue was purified by column (EtOAc) to give 1-[3-(1-phenyl-1,2,4-triazol-3-yl)pyrazin-2-yl]ethenone (480 mg, 70% yield) as a yellow solid.

¹H-NMR (400 MHz, MeOH-d₄): δ=9.18 (s, 1H), 8.84 (d, J=2.26 Hz, 1H), 8.74 (d, J=2.26 Hz, 1H), 7.86 (d, J=8.16 Hz, 2H), 7.58 (t, J=7.78 Hz, 2H), 7.44-7.50 (m, 1H), 2.74 (s, 3H).

### Step 7: Preparation of 1-[3-(1-phenyl-1,2,4-triazol-3-yl)pyrazin-2-yl]ethanamine

To a solution of 1-[3-(1-phenyl-1,2,4-triazol-3-yl)pyrazin-2-yl]ethenone (450 mg, 1.70 mmol) in MeOH (30 mL) was added NH₃ in MeOH (7M, 6 mL) and NH₄OAc (1.308 g, 17.0 mmol) at 15° C. The reaction mixture was stirred for 3 h. Then the mixture was heated to 50° C. and stirred for 16 h. LC-MS showed the reaction was completed. The mixture was quenched with water (90 mL) and extracted with EtOAc (3×50 mL). The combined organic phase was washed with brine (50 mL) dried over Na₂SO₄, filtered and concentrated under reduced pressure to give 1-[3-(1-phenyl-1,2,4-triazol-3-yl)pyrazin-2-yl]ethanamine (400 mg, crude) as a yellow solid.

¹H-NMR (400 MHz, DMSO-d₆): δ=9.51 (s, 1H), 8.73-8.79 (m, 1H), 8.68 (d, J=2.25 Hz, 1H), 7.94-7.98 (m, 2H), 7.62 (t, J=7.94 Hz, 2H), 7.47 (t, J=7.38 Hz, 1H), 4.77 (q, J=6.63 Hz, 1H), 3.10-3.47 (m, 2H), 1.38 (d, J=6.63 Hz, 3H).

### Step 8: Preparation of 2,6-dichloro-N-[1-[3-(1-phenyl-1,2,4-triazol-3-yl)pyrazin-2-yl]ethyl]pyridine-4-carboxamide [I2-1]

To a solution of 1-[3-(1-phenyl-1,2,4-triazol-3-yl)pyrazin-2-yl]ethanamine (433 mg, 2.53 mmol) in ACN (4 mL) was added NMI (370 mg, 4.506 mmol) and TCFH (631 mg, 2.253 mmol) at 15° C. A solution of 2,6-dichloropyridine-4-carboxylic acid (400 mg, 1.502 mmol) in ACN (4 mL) was added. The mixture was stirred at 15° C. for 16 h. LC-MS showed the reaction was completed. The mixture was concentrated under reduced pressure and purified by prep-HPLC (NH₄HCO₃) to give 2,6-dichloro-N-[1-[3-(1-phenyl-1,2,4-triazol-3-yl)pyrazin-2-yl]ethyl]pyridine-4-carboxamide (12-1, 102 mg, 15% yield) as a yellow solid.

¹H-NMR (400 MHz, CDCl₃): δ=8.74-8.81 (m, 2H), 8.67 (d, J=2.26 Hz, 1H), 7.99 (br d, J=7.65 Hz, 1H), 7.82 (d, J=7.65 Hz, 2H), 7.65 (s, 2H), 7.59 (t, J=7.84 Hz, 2H), 7.45-7.51 (m, 1H), 6.54-6.64 (m, 1H), 1.68 (d, J=6.53 Hz, 3H).

### Example 3: Preparation of 3-chloro-N-[1-[3-[1-[(4-methoxyphenyl)methyl]-1,2,4-triazol-3-yl]pyrazin-2-yl]ethyl]-5-(trifluoromethyl)benzamide[11-4]

### Step 1: Preparation of 1-[3-[1-[(4-methoxyphenyl)methyl]-1,2,4-triazol-3-yl]pyrazin-2-yl]ethanamine

To a solution of 1-[3-[1-[(4-methoxyphenyl)methyl]-1,2,4-triazol-3-yl]pyrazin-2-yl]ethanone and 1-[3-[2-[(4-methoxyphenyl)methyl]-1,2,4-triazol-3-yl]pyrazin-2-yl]ethanone (10 g, 32.4 mmol) in MeOH (1 L) was added NH₃ in MeOH (7M, 100 mL) and NH₄OAc (25 g, 324 mmol) at 15° C. The reaction mixture was stirred for 30 min. Then the mixture was heated to 50° C. and stirred for 16 h.

LC-MS showed the reaction was completed. The reaction mixture was concentrated under reduced pressure and purified by HPLC to give a mixture of 1-[3-[1-[(4-methoxyphenyl)methyl]-1,2,4-triazol-3-yl]pyrazin-2-yl]ethanamine and 1-[3-[2-[(4-methoxyphenyl)methyl]-1,2,4-triazol-3-yl]pyrazin-2-yl]ethanamine (10 g, 50% yield) as a yellow oil.

### Step 2: Preparation of 3-chloro-N-[1-[3-[1-[(4-methoxyphenyl)methyl]-1,2,4-triazol-3-yl]pyrazin-2-yl]ethyl]-5-(trifluoromethyl)benzamide (11-4)

To a solution of 3-chloro-5-(trifluoromethyl)benzoic acid (10.8 g, 48.3 mmol) in DMF (50 mL) was added TEA (6.5 g, 64.4 mmol) and HATU (18.4 g, 48.4 mmol) at 0° C. under N₂-atmosphere. The reaction mixture was stirred for 30 min at this temperature. A mixture of 1-[3-[1-[(4-methoxyphenyl)methyl]-1,2,4-triazol-3-yl]pyrazin-2-yl]ethanamine and 1-[3-[2-[(4-methoxyphenyl)methyl]-1,2,4-triazol-3-yl]pyrazin-2-yl]ethanamine (8 g, 32.2 mmol) in DMF (10 mL) was added to the reaction mixture and stirred at 15° C. for 16 h. TLC (EtOAc:EtOH=3:1) showed the reaction was completed. The reaction was quenched with NH₄Cl (aq., 50 mL) and extracted with EtOAc (3×50 mL).

The combined organic phase was washed with brine (3×50 mL), dried over Na₂SO₄, filtered and concentrated under reduced pressure. The residue was purified by column (EtOAc:EtOH=3:1) to give 3-chloro-N-[1-[3-[1-[(4-methoxyphenyl)methyl]-1,2,4-triazol-3-yl]pyrazin-2-yl]ethyl]-5-(trifluoromethyl)benzamide (11-4, 3 g, 18.1% yield) as a yellow oil.

¹H-NMR (400 MHz, CDCl₃): δ=8.71 (d, J=2.38 Hz, 1H), 8.62 (d, J=2.38 Hz, 1H), 8.14 (s, 1H), 7.96-8.04 (m, 3H), 7.74 (s, 1H), 7.35 (d, J=8.66 Hz, 2H), 6.94 (d, J=8.66 Hz, 2H), 6.47-6.57 (m, 1H), 5.43 (s, 2H), 3.83 (s, 3H), 1.60 (d, J=6.65 Hz, 3H).

### Example 4: Preparation of 3-chloro-N-[1-[3-[1-[(2,2-dichlorocyclopropyl)methyl]-1,2,4-triazol-3-yl]pyrazin-2-yl]ethyl]-5-(trifluoromethyl)benzamide[11-14]

### Step 1: Preparation of 3-chloro-N-[1-[3-(1H-1,2,4-triazol-3-yl)pyrazin-2-yl]ethyl]-5-(trifluoromethyl)benzamide[INT]

A solution of a mixture of 3-chloro-N-[1-[3-[1-[(4-methoxyphenyl)methyl]-1,2,4-triazol-3-yl]pyrazin-2-yl]ethyl]-5-(trifluoromethyl)benzamide and 3-chloro-N-[1-[3-[2-[(4-methoxyphenyl)methyl]-1,2,4-triazol-3-yl]pyrazin-2-yl]ethyl]-5-(trifluoromethyl)benzamide (11 g, 21.3 mmol) in TFA (10 mL) was heated to 80° C. and stirred for 16 h. LC-MS showed the reaction was completed.

The reaction mixture was concentrated under reduced pressure und purified by column (EtOAc:EtOH=3:1) to give 3-chloro-N-[1-[3-(1H-1,2,4-triazol-3-yl)pyrazin-2-yl]ethyl]-5-(trifluoromethyl)benzamide (6 g, 71.4% yield) as a gray solid.

¹H-NMR (400 MHz, CDCl₃): δ=8.69 (d, J=2.25 Hz, 1H), 8.65 (d, J=2.25 Hz, 1H), 8.37 (s, 1H), 8.25 (br dd, J=7.00, 1.88 HZ, 1H), 8.03 (s, 1H), 7.99 (s, 1H), 7.75 (s, 1H), 6.51-6.61 (m, 1H) 1.68 (d, J=6.75 Hz, 3H).

LCMS (Method B): (Desired Mass: m/z=396.0; Observed Mass: m/z=397.0) Step 2: Preparation of 3-chloro-N-[1-[3-[1-[(2,2-dichlorocyclopropyl)methyl]-1,2,4-triazol-3-yl]pyrazin-2-yl]ethyl]-5-(trifluoromethyl)benzamide[11-14]

To a solution of 3-chloro-N-[1-[3-(1H-1,2,4-triazol-3-yl)pyrazin-2-yl]ethyl]-5-(trifluoromethyl)benzamide (500 mg, 1.26 mmol) in toluene (10 mL) was added (2,2-dichlorocyclopropyl)methanol (354 mg, 2.5 mmol) and (tributylphosphoranylidene)acetonitrile (600 mg, 2.5 mmol) at 20° C. The reaction mixture was heated to 130° C. and stirred for 12 h. LC-MS showed the reaction was completed. The mixture was concentrated under reduced pressure and purified by prep-HPLC (TFA) to give 3-chloro-N-[1-[3-[1-[(2,2-dichlorocyclopropyl)methyl]-1,2,4-triazol-3-yl]pyrazin-2-yl]ethyl]-5-(trifluoromethyl)benzamide (11-14, 80 mg, 12% yield) as a yellow syrup.

¹H-NMR (400 MHz, CHCl₃-d): 5=8.72 (d, 1H), 8.71 (d, 1H), 8.50 (s, 1H), 8.01-7.96 (m, 3H), 7.72 (s, 1H), 6.54-6.51 (M, 1H), 4.63-4.57 (m, 1H), 4.52-4.46 (m, 1H), 2.34-2.2.30 (m, 1H), 1.89-1.87 (m, 1H), 1.63 (d, 3H), 1.54-1.49 (m, 1H).

### Example 5: Preparation of 3-chloro-N-[1-[3-[1-(3,3-dichloroallyl)-1,2,4-triazol-3-yl]pyrazin-2-yl]ethyl]-5-(trifluoromethyl)benzamide[11-11]

To a solution of 3-chloro-N-[1-[3-(1H-1,2,4-triazol-3-yl)pyrazin-2-yl]ethyl]-5-(trifluoromethyl)benzamide (1 g, 2.52 mmol) in ACN (20 mL) was added 1,1,3-trichloroprop-1-ene (367 mg, 5.04 mmol) and K₂CO₃ (696 mg, 5.04 mmol) at 20° C. The reaction mixture was heated to 50° C. and stirred for 12 h. LC-MS showed the reaction was completed. The reaction was quenched with water (20 mL) and extracted with EtOAc (3×8 mL). The combined organic phase was washed with brine (4×20 mL), dried over Na₂SO₄, filtered and concentrated under reduced pressure. The residue was purified by prep-HPLC (TFA) to give 3-chloro-N-[1-[3-[1-(3,3-dichloroallyl)-1,2,4-triazol-3-yl]pyrazin-2-yl]ethyl]-5-(trifluoromethyl)benzamide (11-11, 200 mg, 16% yield) as a yellow solid.

¹H-NMR (400 MHz, MeOH-d₄): δ=8.66-8.70 (m, 2H), 8.63 (d, J=2.13 Hz, 1H), 8.10 (s, 1H), 8.07 (s, 1H), 7.87 (s, 1H), 6.43 (t, J=6.9 Hz, 1H), 6.25 (q, J=6.88 Hz, 1H), 5.14 (d, H=6.9 Hz, 2H), 1.68 (d, J=6.88 Hz, 3H).

### Example 6: Preparation of 3-chloro-N-[1-[3-(1-isopropyl-1,2,4-triazol-3-yl)pyrazin-2-yl]ethyl]-N-methyl-5-methylsulfonyl-benzamide[11-5]

To a solution of 3-chloro-N-[1-[3-(1-isopropyl-1,2,4-triazol-3-yl)pyrazin-2-yl]ethyl]-5-methylsulfonyl-benzamide (11-7, 300 mg, 0.67 mmol) in DMF (5 mL) was added NaH (24 mg, 1 mmol) portionwise at 0° C. The reaction mixture was stirred for 30 min. Mel (73 mg, 0.52 mmol) was added and the mixture was stirred for 16 h at 15° C. TLC (EtOAc:EtOH=3:1) showed the reaction was completed. The reaction was quenched with water (15 mL) and extracted with EtOAc (3×10 mL). The combined organic phase was washed with brine (3×10 mL), dried over Na₂SO₄, filtered and concentrated under reduced pressure. The residue was purified by prep-HPLC (NH₄HCO₃) to give 3-chloro-N-[1-[3-(1-isopropyl-1,2,4-triazol-3-yl)pyrazin-2-yl]ethyl]-N-methyl-5-methylsulfonyl-benzamide (11-5, 280 mg, 90% yield) as a white solid.

¹H-NMR (400 MHz, DMSO-d₆): δ=8.70 (d, J=2.32 Hz, 1H), 8.67 (d, J=2.20 Hz, 1H), 8.54 (br s, 1H), 7.91 (br s, 1H), 7.55 (br s, 2H), 5.99-6.31 (m, 1H), 4.63 (sept, J=6.7 Hz 1H), 3.22 (s, 3H), 2.87-3.04 (m, 3H), 1.62 (d, J=6.97 Hz, 3H), 1.48 (d, J=6.72 Hz, 6H).

With appropriate modification of the starting materials, the procedures given in the synthesis descriptions were used to obtain further compounds I. The compounds obtained in this manner are listed in the tables that follow, together with physical data.

TABLE I1
compounds of formula I.1
I.1

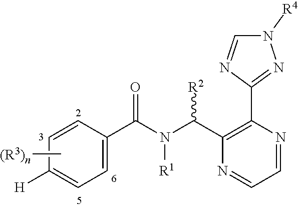

| | | | | | | phys. data | M + H |
|---|---|---|---|---|---|---|---|
| No. | R¹ | R² | R³ | R⁴ | Method | (HPLC RT [min]) | [m/z] |
| I1-1 | H | CH₃ | 3-Br, 5-CF₃ | CH₂CF₃ | A | 1.16 | 524.7 |
| I1-2 | H | CH₃ | 3-(1-CN—cC₃H₄), 5-CF₃ | CH₃ | A | 0.97 | 441.9 |
| I1-3 | CH₂cC₃H₅ | CH₃ | 3-(1-CN—cC₃H₄), 5-CF₃ | CH₃ | B | 1.47 | 496.2 |
| I1-4 | H | CH₃ | 3-Cl, 5-CF₃ | CH₂(4-OCH₃—C₆H₄) | B | 1.60 | 517.2 |
| I1-5 | CH₃ | CH₃ | 3-Cl, 5-S(O)₂CH₃ | CH(CH₃)₂ | B | 1.35 | 463.2 |
| I1-6 | H | CH₃ | 3-Cl, 5-CF₃ | S(O)₂CH₃ | C | 2.58 | 475.0 |
| I1-7 | H | CH₃ | 3-Cl, 5-S(O)₂CH₃ | CH(CH₃)₂ | B | 1.30 | 449.2 |
| I1-8 | H | CH₃ | 3-Cl, 5-CF₃ | CH₂CH═CH₂ | B | 1.50 | 437.2 |
| I1-9 | H | CH₃ | 3-Cl, 5-CF₃ | C₂H₅ | B | 1.70 | 425.2 |
| I1-10 | H | CH₃ | 3,5-Cl₂ | CH₂OCH₃ | B | 1.49 | 407.2 |
| I1-11 | H | CH₃ | 3-Cl, 5-CF₃ | CH₂CH═CCl₂ | B | 1.78 | 505.1 |
| I1-12 | H | CH₃ | 3,5-(CF₃)₂ | CH₃ | A | 1.07 | 444.8 |
| I1-13 | H | CH₃ | 3,5-(CF₃)₂ | CH₂CF₃ | A | 1.17 | 513.2 |
| I1-14 | H | CH₃ | 3-Cl, 5-CF₃ | CH₂(2,2-Cl₂)cC₃H₃ | B | 1.76 | 521.2 |
| I1-15 | H | CH₃ | 3-Br, 5-CF₃ | CH₂C(O)N(CH₃)₂ | B | 1.49 | 528.2 |

TABLE 12
I.2

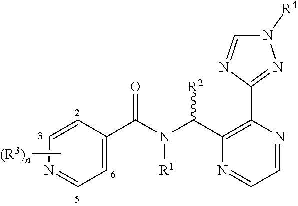

compounds of formula I.2

| No. | R¹ | R² | R³ | R⁴ | Method | phys. data (HPLC RT [min]) | M + H [m/z] |
|---|---|---|---|---|---|---|---|
| I2-1 | H | CH₃ | 3-Cl, 5-Cl | C₆H₅ | B | 1.61 | 440.2 |

### Biological Examples

If not otherwise specified, the test solutions were prepared as follow:

The active compound was dissolved at the desired concentration in a mixture of 1:1 (vol:vol) distilled water:acetone. The test solution was prepared on the day of use.

The activity of the compounds of formula I of the present invention can be demonstrated and evaluated by the following biological tests.

B.1 Green Peach Aphid (Myzus persicae)

For evaluating control of green peach aphid (Myzus persicae) through systemic means, the test unit consisted of 96-well-microtiter plates containing liquid artificial diet under an artificial mem brane.

The compounds were formulated using a solution containing 75% v/v water and 25% v/v DMSO. Different concentrations of formulated compounds were pipetted into the aphid diet, using a custom built pipetter, at two replications.

After application, 5-8 adult aphids were placed on the artificial membrane inside the microtiter plate wells. The aphids were then allowed to suck on the treated aphid diet and incubated at about 23±1° C. and about 50±5% relative humidity for 3 days. Aphid mortality and fecundity was then visually assessed.

In this test, compounds I1-1, I1-2, I1-3, I1-5, I1-6, I1-7, I1-8, I1-9, I1-10, I1-12, I1-13, I1-14, I-15, and I2-1, resp., at 2500 ppm showed at least 75% mortality in comparison with untreated controls.

B.2 Tobacco Budworm (Heliothis virescens)

For evaluating control of tobacco budworm (Heliothis virescens), the test unit consisted of 96-well-microtiter plates containing an insect diet and 15-25 H. virescens eggs.

The compounds were formulated using a solution containing 75% v/v water and 25% v/v DMSO. Different concentrations of formulated compounds were sprayed onto the insect diet at 10 μl, using a custom-built micro atomizer, at two replications.

After application, microtiter plates were incubated at about 28±1° C. and about 80±5% relative humidity for 5 days. Egg and larval mortality was then visually assessed.

In this test, compounds I1-1, I1-2, I1-3, I1-8, I1-9, I1-11, I1-12, I1-13, and I1-14, resp., at 2500 ppm showed at least 75% mortality in comparison with untreated controls.

B.3 Boll Weevil (Anthonomus grandis)

For evaluating control of boll weevil (Anthonomus grandis), the test unit consisted of 96-well-microtiter plates containing an insect diet and 5-10 A. grandis eggs.

The compounds were formulated using a solution containing 75% v/v water and 25% v/v DMSO. Different concentrations of formulated compounds were sprayed onto the insect diet at 5 μl, using a custom-built micro atomizer, at two replications.

After application, microtiter plates were incubated at about 25±1° C. and about 75±5% relative humidity for 5 days. Egg and larval mortality was then visually assessed.

In this test, compounds I1-1, I1-2, I1-3, I1-4, I1-5, I1-6, I1-7, I1-8, I1-9, I1-10, I1-11, I1-12, I1-13, I1-14, I-15, and I2-1, resp., at 2500 ppm showed at least 75% mortality in comparison with untreated controls.

B.4. Southern Armyworm (Spodoptera eridania), 2nd Instar Larvae

The active compounds were formulated by a Tecan liquid handler in 100% cyclohexanone as a 10,000-ppm solution supplied in tubes. The 10,000-ppm solution was serially diluted in 100% cyclohexanone to make interim solutions. These served as stock solutions for which final dilutions were made by the Tecan in 50% acetone:50% water (v/v) into 10 or 20 ml glass vials. A non-ionic surfactant (Kinetic@) was included in the solution at a volume of 0.01% (v/v). The vials were then inserted into an automated electrostatic sprayer equipped with an atomizing nozzle for application to plants/insects. Lima bean plants (variety Sieva) were grown 2 plants to a pot and selected for treatment at the 1st true leaf stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. Ten to 11 armyworm larvae were placed into the bag and the bags zipped closed. Test plants were maintained in a growth room at about 25° C. and about 20-40% relative humidity for 4 days, avoiding direct exposure to fluorescent light (14:10 light:dark photoperiod) to prevent trapping of heat inside the bags. Mortality and reduced feeding were assessed 4 days after treatment, compared to untreated control plants.

In this test, compounds I1-1, I1-2, I1-3, I1-4, I1-5, I1-6, I1-7, I1-8, I1-10, I1-12, I1-13, and I1-14, resp., at 300 ppm at least 75% mortality in comparison with untreated controls.

B.5 Yellow Fever Mosquito (Aedes aegypti)

For evaluating control of yellow fever mosquito (Aedes aegypti) the test unit consisted of 96-well-microtiter plates containing 200 μl of tap water per well and 5-15 freshly hatched A. aegypti larvae.

The active compounds were formulated using a solution containing 75% (v/v) water and 25% (v/v) DMSO. Different concentrations of formulated compounds or mixtures were sprayed onto the insect diet at 2.5 μl, using a custom-built micro atomizer, at two replications.

After application, microtiter plates were incubated at 28±1° C., 80±5% RH for 2 days. Larval mortality was then visually assessed.

In this test, compounds I1-1, I1-2, I1-3, I1-5, I1-6, I1-7, I1-8, I1-9, I1-10, I1-11, I1-12, I1-13, I1-14, I-15, and I2-1, resp., at 2500 ppm showed at least 75% mortality in comparison with untreated controls.

The beneficial activity of the compounds according to the invention with a C-bonded triazole over structurally close compounds known from prior art with N-bonded triazoles was demonstrated by the following comparative experiments:

B.6 ANAPCO (Orchid thrips; Dichromothrips corbetti)

Dichromothrips corbetti adults used for bioassay are obtained from a colony maintained continuously under laboratory conditions. For testing purposes, the test compound is diluted in a 1:1 mixture of acetone:water (vol:vol), plus Kinetic at a rate of 0.01% v/v.

Thrips potency of each compound is evaluated by using a floral-immersion technique. Each orchid petal is dipped into treatment solution and allowed to dry in Petri dishes. Treated petals are placed into individual re-sealable plastic along with about 20 adult thrips. All test arenas are held under dark condition and a temperature of about 28° C. for duration of the assay. The percent mortality is recorded 72 hours after treatment.

B.7 APHICR (Black Bean Aphid; Aphis craccivora)

The active compound is dissolved at the desired concentration in a mixture of 1:1 (vol:vol) distilled water:acetone. Surfactant (Kinetic) is added at a rate of 0.01% (vol/vol). The test solution is prepared at the day of use.

Potted (Kord traditional square pots Size 3″ and 2.5″ in depth) bush beans are cleaned with the apical leaves removed using pointed forceps, leaving only the cotyledon leaves. Plants are inoculated with about 30 mixed-stage aphid colony 24 hours before spraying. Potted bean plants are sprayed with the test solutions using a DeVilbiss hand atomizer (20-30 psi). Treated plants are allowed to air-dry in the laboratory for about an hour before placing them inside the holding room maintained at 27° C., 50% RH and 72 hours light conditions. Stem of the treated bean plants are inserted in the slit of cut circle filter paper to catch the falling dead aphids. Assessment of population reduction (% mortality) is done after 72 hours.

The tables show % mortality in comparison to untreated controls.

| | | B.1 | B.6 | B.7 |
|---|---|---|---|---|
| Structure | Example | 9.9 ppm | 300 ppm | 300 ppm |
| 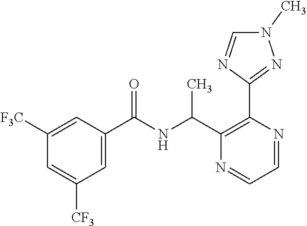 [SMILES: CC(NC(=O)c1cc(C(F)(F)F)cc(C(F)(F)F)c1)c1nccnc1-c1ncn(C)n1] | I1-12 | 100% | 100% | 100% |
| 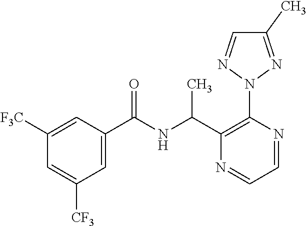 [SMILES: Cc1cnn(-c2nccnc2C(C)NC(=O)c2cc(C(F)(F)F)cc(C(F)(F)F)c2)n1] | WO 2021/037614 P1 | 0% | 0% | 0% |
| 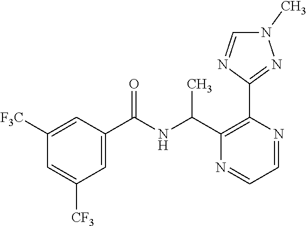 [SMILES: CC(NC(=O)c1cc(C(F)(F)F)cc(C(F)(F)F)c1)c1nccnc1-c1ncn(C)n1] | I1-12 | 100% | 100% | 100% |
| 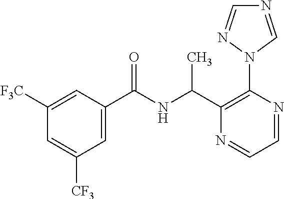 [SMILES: CC(NC(=O)c1cc(C(F)(F)F)cc(C(F)(F)F)c1)c1nccnc1-n1cncn1] | WO 2021/037614 P46 | 25% | 0% | 50% |
| | | B.1 | B.3 | B.6 |
| Structure | Example | 7.9 ppm | 7.9 ppm | 300 ppm |
| 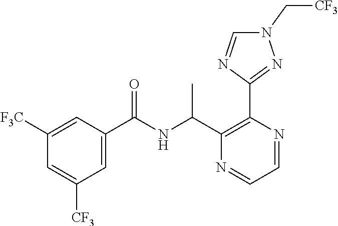 [SMILES: CC(NC(=O)c1cc(C(F)(F)F)cc(C(F)(F)F)c1)c1nccnc1-c1ncn(CC(F)(F)F)n1] | I1-13 | 100% | 100% | 100% |
| 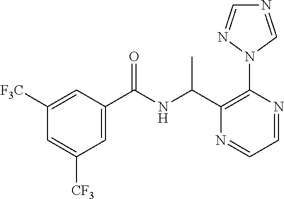 [SMILES: CC(NC(=O)c1cc(C(F)(F)F)cc(C(F)(F)F)c1)c1nccnc1-n1cncn1] | WO 2021/037614 P46 | 0% | 50% | 0% |

1. A compound of formula I 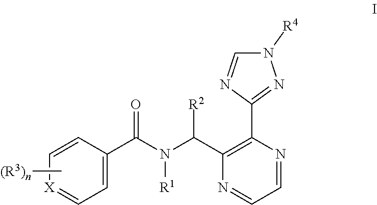
wherein
R¹ is H, OH, C₁-C₆-alkyl, C₁-C₆-haloalkyl, C₃-C₆-cycloalkyl, C₃-C₆-halocycloalkyl, C₁-C₅-alkoxy, C₂-C₄-alkenyl, C₂-C₄-alkynyl, C₁-C₄-alkyl-C₃-C₆-cycloalkyl, C₁-C₄-alkyl-C₃-C₆-halocycloalkyl, which groups are unsubstituted, or partially or fully substituted with R¹¹;
or C(═N—R¹¹)R¹², C(O)R^{11a};
R¹¹ is CN, NO₂, NR¹²R¹³, C(O)NH₂, C(S)NH₂, C(O)OH, OR¹⁴, Si(CH₃)₃; C₁-C₆-alkyl; C₁-C₆-haloalkyl; C₂-C₆-alkenyl; C₂-C₆-haloalkenyl; C₂-C₆-alkynyl; C₂-C₆-haloalkynyl; C₃-C₄-cycloalkyl-C₁-C₂-alkyl, which ring is unsubstituted or substituted with 1 or 2 halogen; 3- to 6-membered heterocyclyl, which rings are unsubstituted or substituted with R^{a}; 5- or 6-membered hetaryl, or phenyl, which rings are unsubstituted or substituted with R^{3a};
R^{a} halogen, CN, NO₂, OH, C₁-C₄-alkyl, C₁-C₄-haloalkyl, C₁-C₄-alkoxy-C₁-C₄-alkyl, C₁-C₄-alkoxy, C₁-C₄-haloalkoxy, C₃-C₄-cycloalkyl, C₃-C₄-halocycloalkyl, S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocycloalkyl, and oxo;
R^{11a} is NR¹²R¹³, C(O)NH₂, C(S)NH₂, C(O)OH, OR¹⁴, Si(CH₃)₃; C₁-C₆-haloalkyl; C₂-C₆-alkenyl; C₂-C₆-haloalkenyl; C₂-C₆-alkynyl; C₂-C₆-haloalkynyl; C₃-C₄-cycloalkyl-C₁-C₂-alkyl, which ring is unsubstituted or substituted with 1 or 2 halogen; 3- to 6-membered heterocyclyl, which rings are unsubstituted or substituted with R^{a};
R¹², R¹³ are independently from each other H, C₁-C₄-alkyl, C₁-C₄-alkoxy, C₁-C₄-haloalkoxy, C₁-C₄-haloalkyl, C₃-C₆-cycloalkyl, C(O)—C₁-C₄-alkyl, C(O)—C₁-C₄-haloalkyl, C(O)—C₃-C₄-cycloalkyl, C(O)—C₃-C₄-halocycloalkyl, C(O)NR¹²¹R¹³¹, S(O)ₘ—C₁-C₄-haloalkyl, S(O)ₘ—C₃-C₄-cycloalkyl, S(O)ₘ—C₃-C₄-halocycloalkyl; 3- to 6-membered heterocyclyl, which rings are unsubstituted or substituted with R^{a}; 5- or 6-membered hetaryl, or phenyl, which rings are unsubstituted or substituted with R^{3a}; or
R¹² and R¹³, together with the nitrogen atom to which they are bound, form a 3-, 4-, 5-, 6- or 7-membered saturated, partially unsaturated or fully unsaturated heterocycle, which heterocycle may additionally contain 1 or 2 heteroatoms or heteroatom groups selected from N, O, and S(O)ₘ as ring members, and which heterocycle is unsubstituted or substituted with one or more substituents selected from R^{a};
R¹²¹ and R¹³¹ are independently from each other H, C₁-C₄-alkyl, C₁-C₄-haloalkyl, C₃-C₆-cycloalkyl, C₃-C₆-halocycloalkyl, C₁-C₄-alkoxy, C₁-C₄-haloalkoxy; C₁-C₄-alkyl-phenyl, C₁-C₄-alkyl-3-6-membered hetaryl, phenyl, 3- to 6-membered heterocyclyl, which rings are unsubstituted or substituted with R^{a}; or 5- or 6-membered hetaryl which rings are unsubstituted or substituted with R^{3a}; or
R¹²¹ and R¹³¹ together with the nitrogen atom they are bound to form a 3-6 membered saturated, partially or fully unsaturated heterocycle, which may further contain 1 or 2 heteroatoms ring members selected from N, O and S, wherein S may be oxidized, which heterocycle is unsubstituted or substituted with R^{a};
m is 0, 1, or 2;
R¹⁴ is H, C₁-C₄-alkyl, C₁-C₄-haloalkyl, C₃-C₆-cycloalkyl, C₃-C₆-halocycloalkyl, C₃-C₄-cycloalkyl-C₁-C₂-alkyl, C₃-C₄-halocycloalkyl-C₁-C₂-alkyl, C(O)—C₁-C₄-alkyl, C(O)—C₁-C₄-haloalkyl, C(O)—C₃-C₄-cycloalkyl, C(O)—C₃-C₄-halocycloalkyl, or phenyl which is unsubstituted or partially or fully substituted with R^{3a};
R² is H, CN, C₁-C₃-alkyl, C₁-C₃-haloalkyl, C₂-C₃-alkenyl, C₂-C₃-alkynyl;
X is CH, CR³, or N;
R³ is halogen, CN, NO₂, C₁-C₄-alkyl, C₃-C₆-cycloalkyl, C₁-C₆-haloalkyl, C₁-C₆-halocycloalkyl, OR¹⁴, S(O)ₘ—R¹⁴; which are unsubstituted or substituted with R^{3a};
R^{3a} halogen, CN, NO₂, OH, C₁-C₄-alkyl, C₁-C₄-haloalkyl, C₁-C₄-alkoxy-C₁-C₄-alkyl, C₁-C₄-alkoxy, C₁-C₄-haloalkoxy, C₃-C₄-cycloalkyl, C₃-C₄-halocycloalkyl, S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocycloalkyl;
n is 0, 1, 2, or 3;
R⁴ is OR¹⁴, CN, C₁-C₆-alkyl, C₃-C₆-cycloalkyl, C₁-C₆-haloalkyl, C₁-C₆-halocycloalkyl, C₂-C₄-alkenyl, C₂-C₄-halo¬alkenyl, C₂-C₄-alkynyl, each unsubstituted or partially or fully substituted with R⁴¹;
S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocyclo¬alkyl, NR¹²R¹³, C(O)NR¹²R¹³, C(O)OR¹⁴, 3- to 6-membered heterocyclyl, which rings are unsubstituted or substituted with R^{a}; 5- or 6-membered hetaryl, or phenyl, which rings are unsubstituted or partially or fully substituted with R³;
R⁴¹ is H, OR¹⁵, NR¹²R¹³, C₁-C₄-alkyl, C₁-C₄-haloalkyl, C₃-C₆-cycloalkyl, C(O)—C₁-C₄-alkyl, C(O)—C₁-C₄-haloalkyl, C(O)—C₃-C₄-cycloalkyl, C(O)—C₃-C₄-halocycloalkyl, C(O)NR¹²¹R¹³¹;
S(O)ₘ—C₁-C₄-haloalkyl, S(O)ₘ—C₃-C₄-cycloalkyl, S(O)ₘ—C₃-C₄-halocycloalkyl; 3- to 6-membered heterocyclyl, 5- or 6-membered hetaryl, or phenyl;
which non-aromatic cyclic R⁴¹ groups are unsubstituted or partially or fully substituted with R^{a};
which aromatic R⁴¹ groups are unsubstituted or partially or fully substituted with R^{3a};
R¹⁵ is H, C₁-C₄-alkyl, or C₁-C₄-haloalkyl, C₃-C₆-cycloalkyl, C₁-C₆-halocycloalkyl, which carbon chains are unsubstituted or partially or fully substituted with R¹¹; or 3- to 6-membered heterocyclyl, which rings are unsubstituted or substituted with R^{a}; 5- or 6-membered hetaryl, or phenyl, which rings are unsubstituted or partially or fully substituted with R^{3a};
and N-oxides, stereoisomers, and agriculturally or veterinarily acceptable salts thereof.

2. The compound of formula I according to claim 1, wherein
R¹¹ is CN, NO₂, NR¹²R¹³, C(O)NH₂, C(S)NH₂, C(O)OH, OR¹⁴, Si(CH₃)₃; C₁-C₆-alkyl; C₁-C₆-haloalkyl; C₂-C₆-alkenyl; C₂-C₆-haloalkenyl; C₂-C₆-alkynyl; C₂-C₆-haloalkynyl; C₃-C₄-cycloalkyl-C₁-C₂-alkyl, which ring is unsubstituted or substituted with 1 or 2 halogen; 3- to 6-membered heterocyclyl, 5- or 6-membered hetaryl, or phenyl, which rings are unsubstituted or substituted with halogen, C₁-C₃-haloalkyl, and/or CN;
R^{11a} is NR¹²R¹³, C(O)NH₂, C(S)NH₂, C(O)OH, OR¹⁴, Si(CH₃)₃; C₁-C₆-haloalkyl; C₂-C₆-alkenyl; C₂-C₆-haloalkenyl; C₂-C₆-alkynyl; C₂-C₆-haloalkynyl; C₃-C₄-cycloalkyl-C₁-C₂-alkyl, which ring is unsubstituted or substituted with 1 or 2 halogen; 3- to 6-membered heterocyclyl, which rings are unsubstituted or substituted with halogen, C₁-C₃-haloalkyl, and/or CN;
R¹², R¹³ are independently from each other H, C₁-C₄-alkyl, C₁-C₄-alkoxy, C₁-C₄-haloalkoxy, C₁-C₄-haloalkyl, C₃-C₆-cycloalkyl, C(O)—C₁-C₄-alkyl, C(O)—C₁-C₄-haloalkyl, C(O)—C₃-C₄-cycloalkyl, C(O)—C₃-C₄-halocycloalkyl, C(O)NR¹²¹R¹³¹, S(O)ₘ—C₁-C₄-haloalkyl, S(O)ₘ—C₃-C₄-cycloalkyl, S(O)ₘ—C₃-C₄-halocycloalkyl; 3- to 6-membered heterocyclyl, 5- or 6-membered hetaryl, or phenyl, which rings are unsubstituted or substituted with halogen, C₁-C₃-haloalkyl, and/or CN;
R¹²¹ and R¹³¹ are independently from each other hydrogen, C₁-C₄-alkyl, C₁-C₄-haloalkyl, C₃-C₆-cycloalkyl, C₃-C₆-halocycloalkyl, C₁-C₄-alkoxy, C₁-C₄-haloalkoxy; C₁-C₄-alkyl-phenyl, C₁-C₄-alkyl-3-6-membered hetaryl, phenyl, 3- to 6-membered heterocyclyl or 5- or 6-membered hetaryl which rings are unsubstituted or substituted with halogen, C₁-C₃-haloalkyl, and/or CN; or
R¹²¹ and R¹³¹ together with the nitrogen atom they are bound to form a 3-6 membered saturated, partially or fully unsaturated heterocycle, which may further contain 1 or 2 heteroatoms ring members selected from N, O and S, wherein S may be oxidized, which heterocycle is unsubstituted or substituted with halogen, C₁-C₃-haloalkyl, and/or CN;
R¹⁴ is H, C₁-C₄-alkyl, C₁-C₄-haloalkyl, C₃-C₆-cycloalkyl, C₃-C₆-halocycloalkyl, C₃-C₄-cycloalkyl-C₁-C₂-alkyl, C₃-C₄-halocycloalkyl-C₁-C₂-alkyl, C(O)—C₁-C₄-alkyl, C(O)—C₁-C₄-haloalkyl, C(O)—C₃-C₄-cycloalkyl, C(O)—C₃-C₄-halocycloalkyl, or phenyl which is unsubstituted or partially or fully substituted with R³;
R² is H, CN, C₁-C₃-alkyl, C₁-C₃-haloalkyl, C₂-C₃-alkynyl;
R⁴ is H, OH, CN, C₁-C₆-alkyl, C₃-C₆-cycloalkyl, C₁-C₆-haloalkyl, C₁-C₆-halocycloalkyl, C₂-C₄-alkenyl, C₂-C₄-halo¬alkenyl, C₂-C₄-alkynyl, C₁-C₄-alkoxy, C₁-C₄-haloalkoxy, each unsubstituted or partially or fully substituted with R⁴¹;
S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocyclo¬alkyl, NR¹²R¹³, C(O)NR¹²R¹³, C(O)OR¹⁴, 3- to 6-membered heterocyclyl, 5- or 6-membered hetaryl, or phenyl, which rings are unsubstituted or partially or fully substituted with R³;
R⁴¹ is H, OR¹⁵, NR¹²R¹³, C₁-C₄-alkyl, C₁-C₄-haloalkyl, C₃-C₆-cycloalkyl, C(O)—C₁-C₄-alkyl, C(O)—C₁-C₄-haloalkyl, C(O)—C₃-C₄-cycloalkyl, C(O)—C₃-C₄-halocycloalkyl, C(O)NR¹²¹R¹³¹; S(O)ₘ—C₁-C₄-haloalkyl, S(O)ₘ—C₃-C₄-cycloalkyl, S(O)ₘ—C₃-C₄-halocycloalkyl; 3- to 6-membered heterocyclyl, 5- or 6-membered hetaryl, or phenyl;
which cyclic R⁴¹ groups are unsubstituted or partially or fully substituted with halogen, C₁-C₃-haloalkyl, and/or CN;
R¹⁵ is H, C₁-C₄-alkyl, or C₁-C₄-haloalkyl, C₃-C₆-cycloalkyl, C₁-C₆-halocycloalkyl, which carbon chains are unsubstituted or partially or fully substituted with R¹¹; or 3- to 6-membered heterocyclyl, 5- or 6-membered hetaryl, or phenyl, which rings are unsubstituted or partially or fully substituted with R³;
and the N-oxides, stereoisomers, and agriculturally or veterinarily acceptable salts thereof.

3. The compound of formula I according to claim 1, wherein R¹ is H.

4. The compound of formula I according to claim 1, wherein R² is CH₃.

5. The compound of formula I according to claim 1, wherein R³ is halogen, CN, C₁-C₄-haloalkyl, C₁-C₄-haloalkoxy, C₃-C₄-cycloalkyl unsubstituted or substituted with one or more CN, C₃-C₄-halocycloalkyl, S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocycloalkyl, or S(O)ₘ—R¹⁴, wherein R¹⁴ is phenyl, which is partially substituted with R^{3a}.

6. The compound of formula I according to claim 1, wherein n is 2 and R³ is in positions 3 and 5.

7. The compound of formula I according to claim 1, wherein X is CH.

8. The compound of formula I according to claim 1, wherein X is N.

9. The compound of formula I according to claim 1, wherein R⁴ is H, C₁-C₃-alkyl, C₁-C₃-haloalkyl, C₁-C₄-alkoxy-C₁-C₄-alkyl, CH₂C(O)NH—C₁-C₆-alkyl, S(O)ₘ—C₁-C₄-alkyl, or phenyl unsubstituted or substituted with one or more groups R³ selected from the group consistinq of halogen, CN, C₁-C₄-haloalkyl, C₁-C₄-haloalkoxy, C₃-C₄-cycloalkyl unsubstituted or substituted with one or more CN, C₃-C₄-halocycloalkyl, S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocycloalkyl, or S(O)ₘ—R¹⁴, wherein R¹⁴ is phenyl, which is partially substituted with R^{3a}.

10. The compound of formula I according to claim 1, which is mainly isomer I.A.

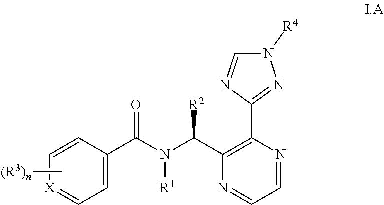

11. An intermediate compound of formula Int, wherein the variables are defined for formula I according to claim 1.

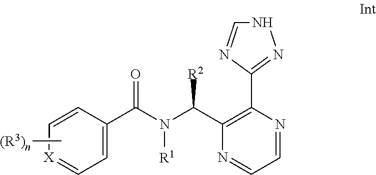

12. An agricultural or veterinary composition comprising at least one compound according to claim 1 and/or at least one agriculturally or veterinarily acceptable salt thereof, and at least one inert liquid and/or solid agriculturally or veterinarily acceptable carrier.

13. An agricultural composition for combating animal pests comprising at least one compound as defined in claim 1 and at least one inert liquid and/or solid acceptable carrier and, if desired, at least one surfactant.

14. A method for combating or controlling invertebrate pests, comprising contacting said pest or its food supply, habitat or breeding grounds with a pesticidally effective amount of at least one compound as defined in claim 1.

15. A method for protecting growing plants from attack or infestation by invertebrate pests, comprising contacting a plant, or soil or water in which the plant is growing, with a pesticidally effective amount of at least one compound as defined in claim 1.

16. A seed comprising a compound as defined in claim 1, or enantiomers, diastereomers, or salts thereof, in an amount of from 0.1 g to 10 kg per 100 kg of seed.

17. A method for treating or protecting an animal from infestation or infection by an invertebrate pest comprising bringing the animal in contact with a pesticidally effective amount of at least one compound of the formula I as defined in claim 1, a stereoisomer thereof, and/or at least one veterinarily acceptable salt thereof.
